SEQUENCE LISTING (9 sequences)

SEQ ID NO: 1 - DNA, 20 bp, Artificial Sequence
  primer GAPDH capture
ttactccttggaggccatgt

SEQ ID NO: 2 - DNA, 20 bp, Artificial Sequence
  primer GAPDH capture
atggtgaaggtcggtgtcaa

SEQ ID NO: 3 - DNA, 20 bp, Artificial Sequence
  primer BRCA1 capture
ttgggagggggctcgggcat

SEQ ID NO: 4 - DNA, 20 bp, Artificial Sequence
  primer BRCA1 capture
cagagctggcagcggacggt

SEQ ID NO: 5 - DNA, 20 bp, Artificial Sequence
  primer Histone H4 capture
aatccgcgatgcagttacct

SEQ ID NO: 6 - DNA, 18 bp, Artificial Sequence
  primer Histone H4 capture
agtgtcggtacctgcacc

SEQ ID NO: 7 - DNA, 20 bp, Artificial Sequence
  primer GAPDH capture, 1 mismatch
ttactccttagaggccatgt

SEQ ID NO: 8 - DNA, 20 bp, Artificial Sequence
  primer GAPDH capture, 2 mismatches
ttattccttggaggccatat

SEQ ID NO: 9 - DNA, 20 bp, Artificial Sequence
  primer GAPDH capture, 3 mismatches
ttattccttagaggccatat

(19) United States
(12) Patent Application Publication
(10) Pub. No.: US 2010/0133117 A1
(43) Pub. Date: Jun. 3, 2010

(54) METHODS FOR ELECTROCHEMICAL DETECTION/QUANTIFICATION OF A NUCLEIC ACID

(75) Inventor: Zhiqiang Gao, Singapore (SG)

Correspondence Address: INTELLECTUAL PROPERTY / TECHNOLOGY LAW, PO BOX 14329, RESEARCH TRIANGLE PARK, NC, 27709, US

(73) Assignee: AGENCY FOR SCIENCE, TECHNOLOGY AND RESEARCH, (SG)

(21) Appl. No.: 12/376,782

(22) PCT Filed: Aug. 8, 2006
(86) PCT No.: PCT/SG2006/000226

§ 371 Date: Feb. 7, 2009

(51) Int. Cl. C12Q 1/28

(52) U.S. Cl. 205/777.5; 205/780.5; 204/403.01; 204/403.14

(57) ABSTRACT

The invention provides methods and kits for the electrochemical detection and/or quantification of a target nucleic acid molecule by means of a detection electrode. In one method there is immobilized on the detection electrode a peptide nucleic acid (PNA) capture molecule, which has a nucleotide sequence that is at least partially complementary to at least a portion of the target nucleic acid molecule. The electrode is contacted with a solution expected to include the target nucleic acid molecule and the target nucleic acid molecule allowed to hybridize to the PNA, thereby allowing the formation of a complex between the PNA capture molecule and the target nucleic acid molecule. A polymerisable positively chargeable precursor is added, which associates to the complex formed between the PNA capture molecule and the target nucleic acid molecule. A suitable reactant molecule is added, initiating the polymerisation of the polymerisable positively chargeable precursor and the formation of an electroconductive polymer. An electrochemical measurement at the detection electrode is performed. In another method a nucleic acid capture molecule is immobilized on the electrode. In this method a polymerisable positively chargeable precursor, a suitable substrate molecule, and an enzyme attached to a detection probe nucleic acid molecule are used. The detection probe nucleic acid molecule is at least partially complementary to at least a portion of the target nucleic acid molecule. The detection probe hybridizes to a portion of the target nucleic acid that is different from the portion to which the capture nucleic acid molecule hybridises.

The present invention relates to methods for the electrochemical detection and/or quantification of a target nucleic acid molecule.

The detection and quantification of nucleic acids is a fundamental method not only in analytical chemistry but also in biochemistry, food technology or medicine. The most frequently used methods for determining the presence and concentration of nucleic acids include the detection by autoradiography, fluorescence, chemiluminescence or bioluminescence as well as electrochemical techniques. Electrochemical techniques include conductivity measurements (Park, S. J., et al., Science (2002) 295, 1503-1506; US patent application 2005/0079533), nucleic acid-intercalation methods (Zeman, S. M., et al., Proc. Natl. Acad. Sci. USA (1998) 95, 11561-11565; Erkkila, K. E. et al., Chem. Rev. (1999) 99, 2777-2795), and detection by catalytic amplification (Caruana, D. J., & Heller, A. J., J. Am. Chem. Soc. (1999) 121, 769-774; Patolsky, F. et al., Angew. Chem. Int. Ed. (2002) 41, 3398-3402). Conductivity measurements can for instance be based on an oligonucleotide functionalised with a gold nanoparticle (Park et al., 2002, supra) or with a conductive polymer (US patent application 2005/0079533).

A technique for the specific detection of a selected nucleic acid well established in the art is based on the hybridisation between a nucleic acid capture probe and a target nucleic acid. Typically the respective nucleic acid capture probe is immobilized onto a solid support, and subsequently one of the above mentioned detection methods is employed.

Sensitivity and selectivity are the two important issues being constantly addressed in the evaluation of nucleic acid detection systems. The most popular methods are performed using fluorescent tags in array formats in conjunction with solution phase (off-chip) pre-amplification/labelling approaches employing polymerase chain reactions. They offer the highest degree of sensitivity, the highest throughput, and the widest dynamic range. However, the amplification power of polymerase chain reactions may be dramatically affected by small variations in experimental conditions and sample compositions. Optimisation of the complicated primers and experimental conditions for each specific gene is a formidable task. Often, the finalized amplification protocols are not optimal for many genes being studied. PCR-based amplification methods therefore do not faithfully reproduce the relative concentrations of genes in complex matrixes, due to selective and nonlinear target amplifications (Ho, H. A., et al., J. Am. Chem. Soc. (2005) 127, 12673-12676). Moreover, optical transduction engages not only highly precise and expensive equipment but also complex algorithms to interpret the data. Furthermore, off-chip target amplifications also significantly increase the cost of the procedures and often lead to sequence-dependent quantification bias.

Consequently, in situ signal amplification strategies, such as rolling circle amplification (RCA), T7 DNA polymerase, branched DNA technology, catalysed reporter deposition, dendritic tags, enzymatic amplification and chemical amplification, have been proposed for both optical and electrochemical systems. Among them, the inherent miniaturization of electrochemical devices, excellent compatibility with advanced semiconductor technology, and low cost make electronic transduction-based biosensors very promising candidates for analysing nucleic acids with reduced cost and size of the read-out unit in comparison with the more conventional optical systems. It has been shown that the sensitivity of those assays is comparable to that of PCR-based fluorescent assays. As few as 10³ copies of genes can be directly detected (Zhang, Y., et al., Anal. Chem. (2003) 75, 3267-3269; Xie, H., et al., Anal. Chem. (2004) 76, 1611-1617). While such improvements may enhance the sensitivity of nucleic acid detection, inherent disadvantages are additional costs for amplification reagents such as primers, as well as the additional time required for signal amplification.

Thus, there remains a need for an alternative method for the detection of nucleic acids, which overcomes the above limitations and allows the detection even of picomolar levels of a nucleic acid with high sensitivity.

Accordingly it is an object of the present invention to provide methods for the detection and/or quantification of a nucleic acid, which avoids the discussed signal amplification strategies.

According to a first aspect, the invention provides methods for the electrochemical detection and/or quantification of a target nucleic acid molecule by means of a detection electrode.

A first respective method includes providing a detection electrode. The method further includes providing a peptide nucleic acid (PNA) capture molecule. The PNA capture molecule has a nucleotide sequence that is at least partially complementary to at least a portion of the target nucleic acid molecule. The method further includes immobilizing on the detection electrode the PNA capture molecule. The method further includes contacting the electrode with a solution expected to include the target nucleic acid molecule. The method also includes allowing the target nucleic acid molecule to hybridise to the PNA capture molecule on the electrode, thereby allowing the formation of a complex between the PNA capture molecule and the target nucleic acid molecule. Furthermore the method includes adding a polymerisable positively chargeable precursor. The polymerisable positively chargeable precursor has an electrostatic net charge that is complementary to the electrostatic net charge of the target nucleic acid molecule. The polymerisable positively chargeable precursor associates to the complex formed between the PNA capture molecule and the target nucleic acid molecule. Furthermore, the polymerisation of the precursor can be carried out by means of a suitable reactant molecule. The method further includes adding a suitable reactant molecule, thereby initiating the polymerisation of the polymerisable positively chargeable precursor. Thereby an electroconductive polymer (i.e. generally a conducting polymer) is formed from the polymerisable precursor. This electroconductive polymer is associated with the complex formed between the PNA capture molecule and the target nucleic acid molecule. Further, the method includes performing an electrochemical measurement at the detection electrode. The method also includes detecting and/or quantifying the presence of the target nucleic acid molecule based on the electrochemical measurement.

According to a particular embodiment, the method includes exposing the reactant molecule to a suitable catalyst. The catalyst may be light, a metal chloride, a metal bromide, a metal sulphate or an enzyme. In such embodiments the reactant molecule may be a substrate molecule for the catalyst.

A second respective method includes providing a detection electrode. The method further includes providing a nucleic acid capture molecule. The nucleic acid capture molecule has a nucleotide sequence that is at least partially complementary to at least a portion of the target nucleic acid molecule. The method also includes immobilizing on the detection electrode the nucleic acid capture molecule. The method further includes contacting the electrode with a solution expected to include the target nucleic acid molecule. The method also includes allowing the target nucleic acid molecule to hybridise to the nucleic acid capture molecule on the electrode, thereby allowing the formation of a complex between the nucleic acid capture molecule and the target nucleic acid molecule. Furthermore the method includes adding a polymerisable positively chargeable precursor. The polymerisable positively chargeable precursor has an electrostatic net charge that is complementary to the electrostatic net charge of the target nucleic acid molecule. The polymerisable positively chargeable precursor associates to the complex formed between the nucleic acid capture molecule and the target nucleic acid molecule. Furthermore, the polymerisation of the precursor can be carried out by means of a suitable enzyme and a substrate molecule. The method further includes adding a suitable substrate molecule. The method also includes adding an enzyme attached to a detection probe nucleic acid molecule. The detection probe nucleic acid molecule is at least partially complementary to at least a portion of the target nucleic acid molecule. The detection probe hybridises to a portion of the target nucleic acid that is different from the portion to which the capture nucleic acid molecule hybridises. Thereby the method includes allowing the detection probe nucleic acid molecule to hybridise to the target nucleic acid molecule. The method thereby also includes catalysing the polymerisation of the polymerisable positively chargeable precursor. An electroconductive polymer is formed from the polymerisable precursor. This electroconductive polymer is associated with the complex formed between the nucleic acid capture molecule and the target nucleic acid molecule. Further, the method includes performing an electrochemical measurement at the detection electrode. The method also includes detecting and/or quantifying the presence of the target nucleic acid molecule based on the electrochemical measurement.

According to a further aspect, the invention provides a kit for the electrochemical detection of a target nucleic acid molecule.

In a first embodiment a kit includes a detection electrode. The respective kit further includes a PNA capture molecule. This PNA capture molecule has a nucleotide sequence that is at least partially complementary to at least a portion of the target nucleic acid molecule. The kit also includes a polymerisable positively chargeable precursor. The electrostatic net charge of the polymerisable positively chargeable precursor is complementary to the electrostatic net charge of the target nucleic acid molecule. Furthermore the kit includes a suitable reactant molecule. In some embodiments the kit also includes a catalyst.

In a second embodiment a kit includes a detection electrode. The respective kit further includes a nucleic acid capture molecule. This nucleic acid capture molecule has a nucleotide sequence that is at least partially complementary to at least a portion of the target nucleic acid molecule. The kit also includes a polymerisable positively chargeable precursor. The electrostatic net charge of the polymerisable positively chargeable precursor is complementary to the electrostatic net charge of the target nucleic acid molecule. Furthermore the kit includes a substrate molecule. The kit also includes an enzyme attached to a probe nucleic acid molecule. The probe nucleic acid molecule is at least partially complementary to at least a portion of the target nucleic acid molecule.

The invention will be better understood with reference to the detailed description when considered in conjunction with the non-limiting examples and the accompanying drawings, in which:

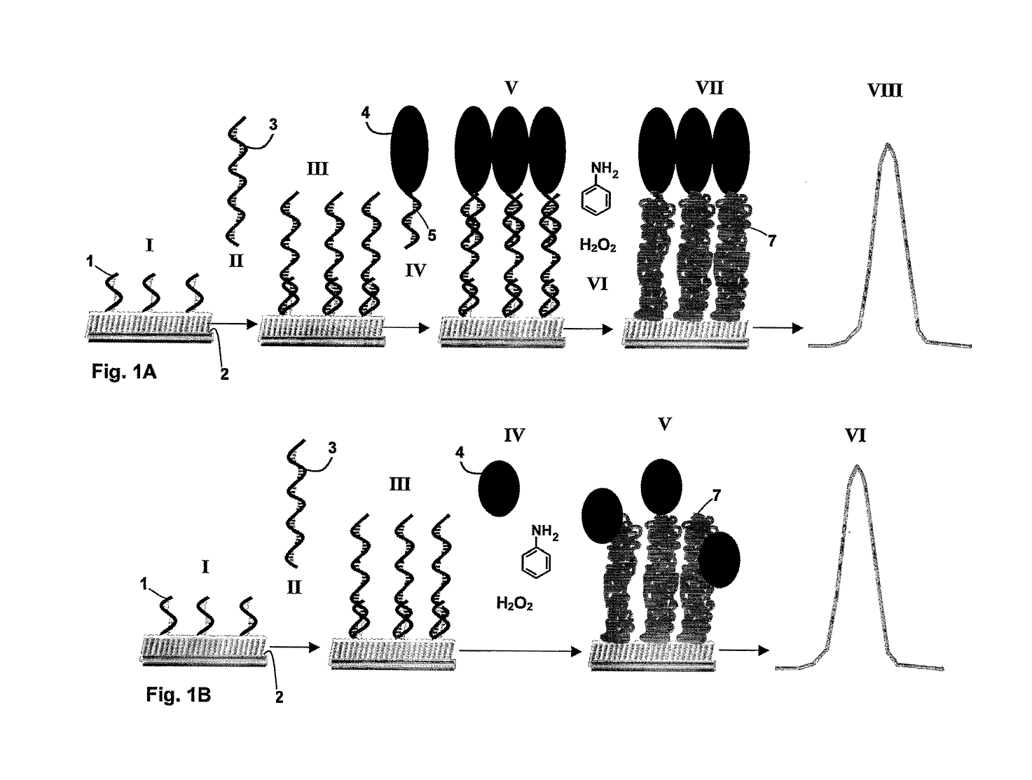

FIG. 1 depicts a schematic representation of exemplary methods of the present invention. FIG. 1A: A nucleic acid capture molecule (1) is immobilized on a detection electrode (I). The electrode is contacted (II) with a solution suspected or known to include a target nucleic acid molecule (3). The two nucleic acid molecules hybridise (III). An enzyme (4) attached to a detection probe nucleic acid molecule (5) is added (IV). The detection probe nucleic acid molecule hybridises to the target nucleic acid molecule at a portion different from the portion where the capture nucleic acid hybridises (V). The polymerisable positively charged precursor molecule aniline and the reactant molecule hydrogen peroxide are added (VI). The positively charged precursor polymerises (VII), forming an electroconductive polymer (7). An electrochemical measurement is performed (VI).

FIG. 1B: A PNA capture molecule (6) is immobilized on a detection electrode (I). The electrode is contacted (II) with a solution suspected or known to include a target nucleic acid molecule (3). The two nucleic acid molecules hybridise (III). The polymerisable positively charged precursor molecule aniline and the reactant molecule hydrogen peroxide, as well as an optional catalyst (4), are added (IV). The positively charged precursor polymerises (V), forming an electroconductive polymer (7). An electrochemical measurement is performed (VI).

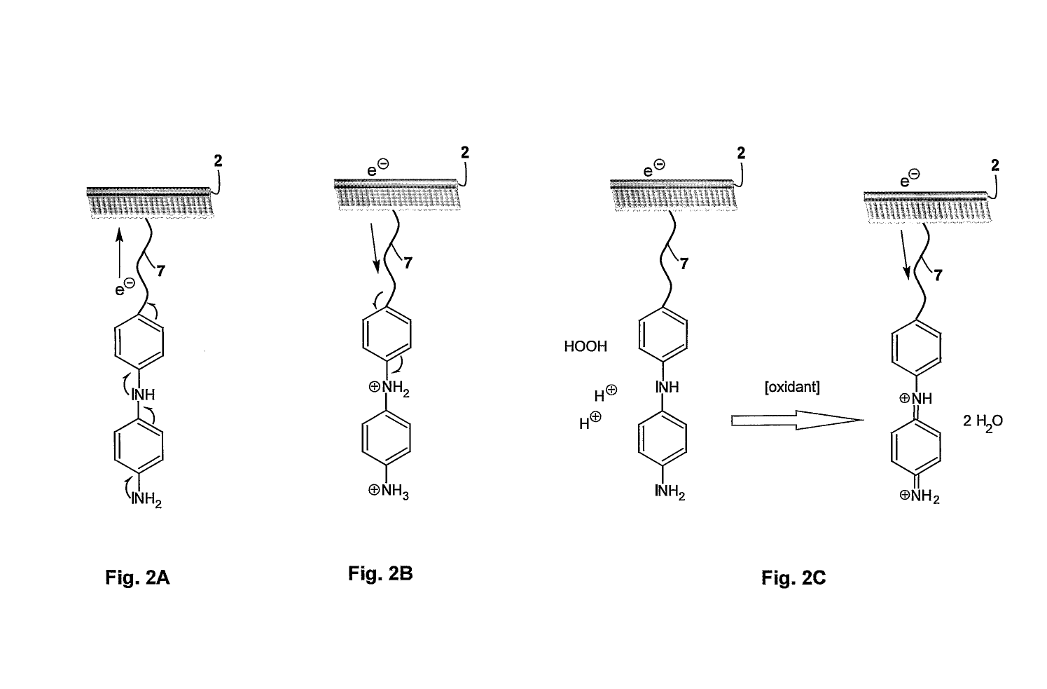

FIG. 2 illustrates possible mechanisms of the electrochemical detection used in the methods of the invention, with aniline selected as an example of a polymerisable positively chargeable precursor. FIG. 2A: The free valence electrons of the amino groups are capable of donating electron density via the aromatic ring systems of the electroconductive polymer (7) polyaniline to the detection electrode (2). FIG. 2B: Protonated amino groups are capable of receiving electron density via the aromatic ring systems of the electroconductive polymer (7) polyaniline from the detection electrode (2). FIG. 2C: The electroconductive polymer (7) polyaniline, conductively associated to the detection electrode (2), is formed by an oxidative polymerisation, during which two electrons are transferred to hydrogen peroxide. One or both of these electrons can be replaced by electrons from the detection electrode during an electrochemical measurement, via the electroconductive polymer.

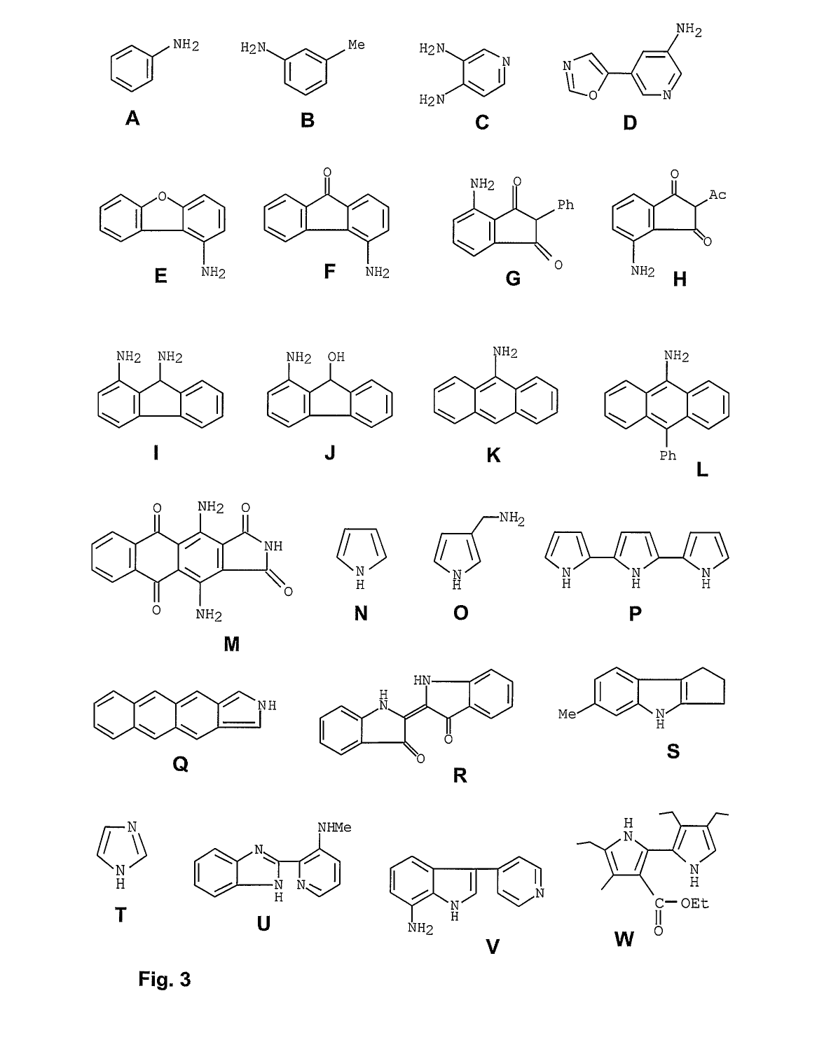

FIG. 3 shows examples of aromatic amines that may serve as a substrate molecule: A: Aniline, Chemical Abstracts No. 62-53-3; B: 3-methylaniline, Chemical Abstracts No. 108-44-1; C: 3,4-pyridinediamine, CAS-No. 54-96-6; D: 5-(5-oxazolyl)-3-pyridinamine, CAS-No. 893566-28-4; E: 1-aminodibenzofuran, CAS-No. 50548-40-8; F: 4-amino-fluoren-9-one, CAS-No. 4269-15-2; G: 4-amino-2-phenyl-indene-1,3(2H)-dione, CAS-No. 6795-96-6; H: 2-acetyl-4-amino-indene-1,3(2H)-dione, CAS-No. 25125-06-8; I: fluorine-1,9-diamine, CAS-No 15824-95-0; J: 1-amino-fluoren-9-ol, CAS-No. 6957-58-0; K: 9-anthraceneamine, CAS-No. 779-03-3; L: 10-phenyl-9-anthraceneamine, CAS-No 1718-54-3; M: 4,11-diamino-naphth[2,3-f]isoindole-1,3,5,10(2H)-tetrone, CAS-No. 128-81-4; N: pyrrole, CAS-No. 109-97-7; O: 3-methanamine-pyrrol, CAS-No. 888473-50-5; P: 2,2′,2″-tripyrrole, CAS-No. 3260-45-5; Q: napht[2,3-f]isoindole, CAS-No. 259-05-4; R: 2-(1,3-dihydro-3-oxo-indol-2-ylidene)-1,2-dihydro-indol-3-one, CAS-No. 482-89-3; S: 1,2,3,4-tetrahydro-6-methyl-cyclopent[b]indole, CAS-No. 887122-89-6; T: imidazole, CAS-No. 288-32-4; U: 2-(benzimidazol-2-yl)-N-methyl-3-pyridinamine, CAS-No. 500857-92-1; V: 3-(4-pyridinyl)-indol-7-amine, CAS-No 887615-82-9; W: 3′,4′,5-triethyl-4-methyl-[2,2′-bi-pyrrole]-3-carboxylic acid ethyl ester, CAS-No. 679816-79-6.

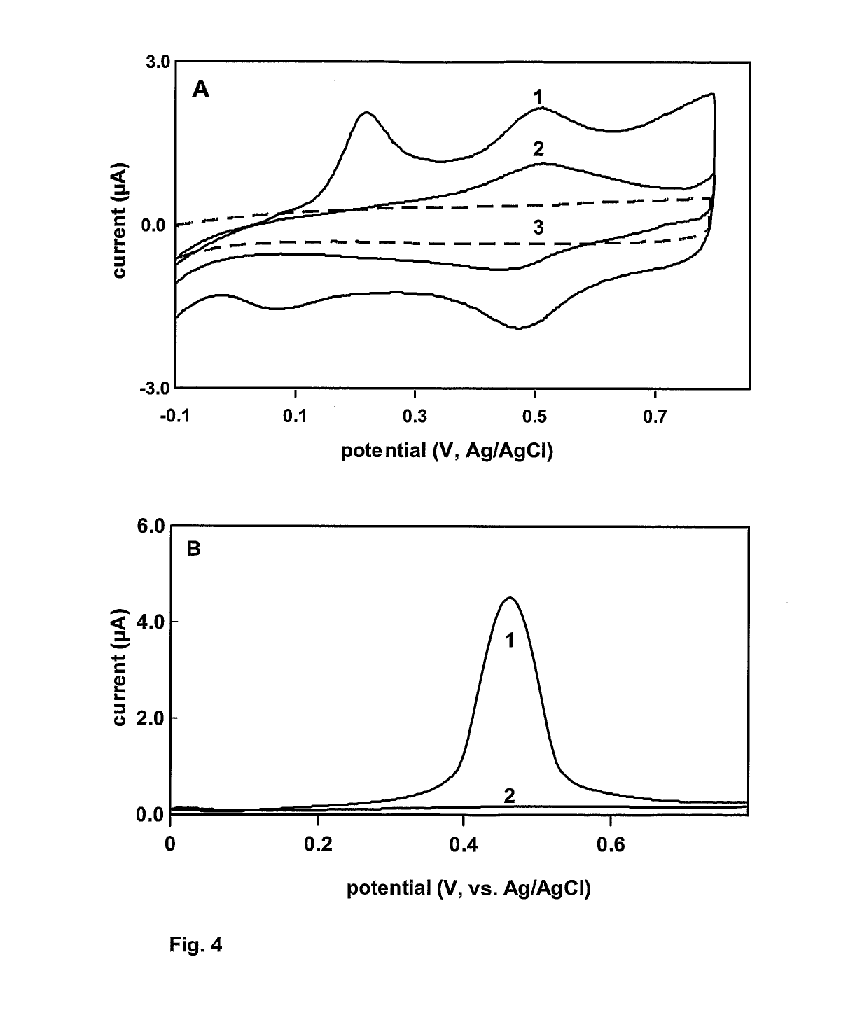

FIG. 4A depicts cyclic voltammograms of polyaniline films prepared by (1) electrodeposition, (2) horse-radish peroxidase catalyzed polyaniline deposition on a detection electrode with GAPDH-specific PNA capture molecule (cf. FIG. 1A), and (3) the detection electrode with GAPDH-specific PNA capture molecule before polyaniline deposition. A 10 min incubation in 30 mM aniline/2.5 mM H₂O₂ in pH 4.0, 0.10 M phosphate buffer was performed. A supporting electrolyte was 1.0 M H₂SO₄, potential scan rate 100 mV/s. FIG. 4B shows square wave voltammetry (SWV) of (1) the detection electrode with PNA capture molecule and (2) control. A supporting electrolyte was 1.0 M H₂SO₄, amplitude 25 mV, step 3 mV, and frequency 15 Hz.

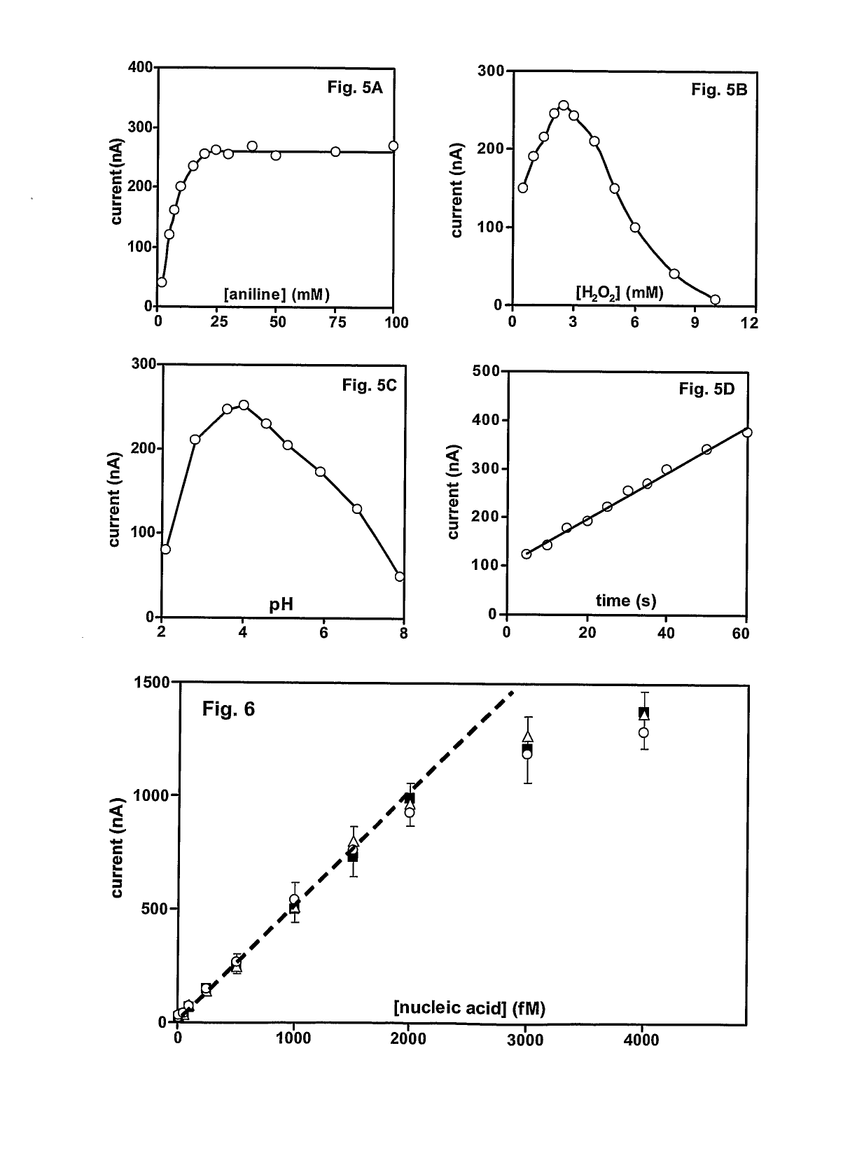

FIG. 5 shows the dependence of the SWV peak current of 250 fM GAPDH on (A) aniline concentration, (B) H₂O₂ concentration, (C) pH, and (D) deposition time. A supporting electrolyte was pH 4.0, 0.10 M phosphate buffer, SWV amplitude 25 mV, step 3 mV, and frequency 15 Hz.

FIG. 6 depicts calibration curves for (o) His4, (V) BRCA1, and (▪) GAPDH. A 30 min incubation in the pH 4.0, 0.10 M phosphate buffer containing 30 mM aniline/2.5 mM H₂O₂ was carried out. A supporting electrolyte was pH 4.0, 0.10 M phosphate buffer, SWV amplitude 25 mV, step 3 mV, and frequency 15 Hz.

FIG. 7 depicts SWV of 100 fM GAPDH at detection electrodes with (1) complementary, (2) one-, (3) two-, and (4) three-base mismatched capture probes. Following hybridization with the detection probe nucleic acid (coupled to horseradish peroxidase), a 30 min incubation in the pH 4.0, 0.10 M phosphate buffer containing 30 mM aniline/2.5 mM H₂O₂ was carried out. A supporting electrolyte was 0.10 M phosphate buffer pH 4.0, SWV amplitude 25 mV, step 3 mV, and frequency 15 Hz.

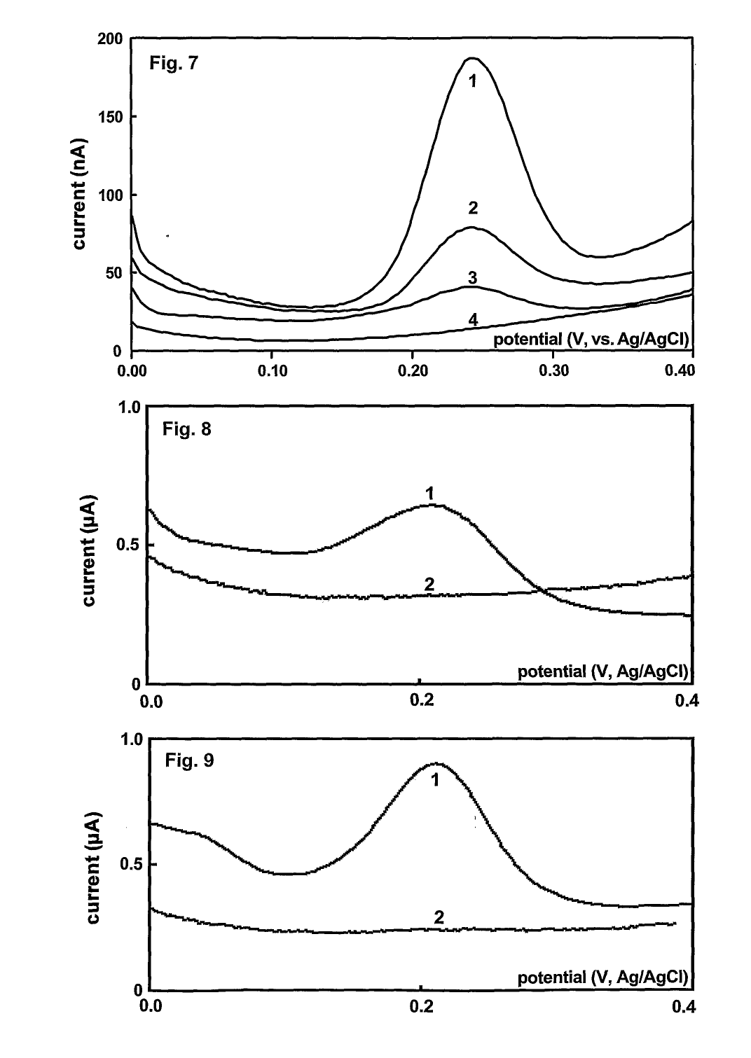

FIG. 8 depicts SWV of 100 fM GAPDH (1) and control (2). The method outlined in FIG. 1B was carried out, using a PNA capture molecule and horse-radish peroxidase as a catalyst. A 30 min incubation in the pH 4.0, 0.10 M phosphate buffer containing 0.025 mg/ml horse radish peroxidase, 30 mM aniline/2.5 mM H₂O₂ was carried out. A supporting electrolyte was pH 4.0, 0.10 M phosphate buffer, SWV amplitude 25 mV, step 3 mV, and frequency 15 Hz.

FIG. 9 depicts SWV of 100 fM GAPDH (1) and control (2). The method outlined in FIG. 1B was carried out, using a PNA capture molecule and ammonium persulfate as an initiator. A 30 min incubation in the pH 4.0, 0.10 M phosphate buffer containing 2 mM NH₄S₂O₈, 30 mM aniline/2.5 mM H₂O₂ was carried out. A supporting electrolyte was pH 4.0, 0.10 M phosphate buffer, SWV amplitude 25 mV, step 3 mV, and frequency 15 Hz.

As will be apparent from the above, the present invention provides methods for the electrochemical detection and/or quantification of a target nucleic acid molecule by means of a detection electrode.

The methods of the invention are based on an electrochemical measurement, such as an amperometric measurement, at a detection electrode. The detection electrode may be of any material as long as an electrochemical detection can be carried out. It may be desired to select the material of the substrate in order to immobilize a nucleic acid thereon (see below). The surface of the detection electrode, or a part thereof, may also be altered, e.g. by means of a treatment carried out to alter characteristics of the solid surface. Such a treatment may include various means, such as mechanical, thermal, electrical or chemical means. As an illustrative example, the surface properties of any hydrophobic surface can be rendered hydrophilic by coating with a hydrophilic polymer or by treatment with surfactants. Examples of a chemical surface treatment include, but are not limited to exposure to hexamethyldisilazane, trimethylchlorosilane, dimethyldichlorosilane, propyltrichlorosilane, tetraethoxysilane, glycidoxypropyltrimethoxy silane, 3-aminopropyltriethoxysilane, 2-(3,4-epoxy cyclohexyl)-ethyltrimethoxysilane, 3-(2,3-epoxy propoxyl)propyltrimethoxysilane, polydimethylsiloxane (PDMS), γ-(3,4-epoxycyclohexyl)ethyltrimethoxysilane, poly(methyl methacrylate) or a polymethacrylate co-polymer, urethane, polyurethane, fluoropolyacrylate, poly(methoxy polyethylene glycol methacrylate), poly(dimethyl acrylamide), poly[N-(2-hydroxypropyl)methacrylamide] (PHPMA), α-phosphorylcholine-o-(N,N-diethyldithio-carbamyl)undecyl oligoDMAAm-oligo-STblock co-oligomer (cf. e.g. Matsuda, T., et al., Biomaterials, (2003), 24, 4517-4527), poly(3,4-epoxy-1-butene), 3,4-epoxy-cyclohexyl-methylmethacrylate, 2,2-bis[4-(2,3-epoxy propoxy)phenyl]propane, 3,4-epoxy-cyclohexyl-methylacrylate, (3′,4′-epoxycyclohexylmethyl)-3,4-epoxycyclohexyl carboxylate, di-(3,4-epoxycyclohexylmethyl)adipate, bisphenol A (2,2-bis-(p-(2,3-epoxy propoxy)phenyl) propane) or 2,3-epoxy-1-propanol.

The methods of the present invention allow for the detection of any target nucleic acid molecule. The term “nucleic acid molecule” as used herein refers to any nucleic acid in any possible configuration, such as single stranded, double stranded or a combination thereof. Nucleic acids include for instance DNA molecules, RNA molecules, analogues of the DNA or RNA generated using nucleotide analogues or using nucleic acid chemistry, locked nucleic acid molecules (LNA), and protein nucleic acids molecules (PNA). DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label.

Many nucleotide analogues are known and can be used in nucleic acids used in the methods of the invention. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

The target nucleic acid molecule may be included in any sample of any origin. It may for instance, but not limited to, be derived from human or non-human animals, plants, bacteria, viruses, spores, fungi, or protozoa, or from organic or inorganic material of synthetic or biological origin. Accordingly, any of the following samples selected from, but not limited to, the group consisting of a soil sample, an air sample, an environmental sample, a cell culture sample, a bone marrow sample, a rainfall sample, a fallout sample, a sewage sample, a ground water sample, an abrasion sample, an archaeological sample, a food sample, a blood sample, a serum sample, a plasma sample, an urine sample, a stool sample, a semen sample, a lymphatic fluid sample, a cerebrospinal fluid sample, a nasopharyngeal wash sample, a sputum sample, a mouth swab sample, a throat swab sample, a nasal swab sample, a bronchoalveolar lavage sample, a bronchial secretion sample, a milk sample, an amniotic fluid sample, a biopsy sample, a cancer sample, a tumour sample, a tissue sample, a cell sample, a cell culture sample, a cell lysate sample, a virus culture sample, a nail sample, a hair sample, a skin sample, a forensic sample, an infection sample, a nosocomial infection sample, a production sample, a drug preparation sample, a biological molecule production sample, a protein preparation sample, a lipid preparation sample, a carbohydrate preparation sample, a space sample, an extraterrestrial sample or any combination thereof may be processed in a method of the invention. Where desired, a respective sample may have been pre-processed to any degree. As an illustrative example, a tissue sample may have been digested, homogenised or centrifuged prior to being used with the device of the present invention. The sample may furthermore have been prepared in form of a fluid, such as a solution. Examples include, but are not limited to, a solution or a slurry of a nucleotide, a polynucleotide, a nucleic acid, a peptide, a polypeptide, an amino acid, a protein, a synthetic polymer, a biochemical composition, an organic chemical composition, an inorganic chemical composition, a metal, a lipid, a carbohydrate, a combinatory chemistry product, a drug candidate molecule, a drug molecule, a drug metabolite or of any combinations thereof. Further examples include, but are not limited to, a suspension of a metal, a suspension of metal alloy, and a solution of a metal ion or any combination thereof, as well as a suspension of a cell, a virus, a microorganism, a pathogen, a radioactive compound or of any combinations thereof. It is understood that a sample may furthermore include any combination of the aforementioned examples. As an illustrative example, the sample that includes the target nucleic acid molecule may be a mammal sample, for example a human or mouse sample, such as a sample of total mRNA.

In some embodiments the sample is a fluid sample, such as a liquid or a gas. In other embodiments the sample is solid. In case of a solid or gaseous sample, an extraction by standard techniques known in the art may be carried out in order to dissolve the target nucleic acid molecule in a solvent. Accordingly, the target nucleic acid molecule, or the expected target nucleic acid molecule, is provided in form of a solution for the use in the present invention. As an illustrative example, the target nucleic acid molecule may be provided in form of an aqueous solution.

Where desired, further matter may be added to the respective solution, for example dissolved or suspended therein. As an illustrative example an aqueous solution may include one or more buffer compounds. Numerous buffer compounds are used in the art and may be used to carry out the various processes described herein. Examples of buffers include, but are not limited to, solutions of salts of phosphate, carbonate, succinate, carbonate, citrate, acetate, formate, barbiturate, oxalate, lactate, phthalate, maleate, cacodylate, borate, N-(2-acetamido)-2-amino-ethanesulfonate (also called (ACES), N-(2-hydroxyethyl)-piperazine-N′-2-ethanesulfonic acid (also called HEPES), 4-(2-hydroxyethyl)-1-piperazine-propanesulfonic acid (also called HEPPS), piperazine-1,4-bis(2-ethanesulfonic acid) (also called PIPES), (2-[Tris(hydroxymethyl)-methylamino]-1-ethansulfonic acid (also called TES), 2-cyclohexyl-amino-ethansulfonic acid (also called CHES) and N-(2-acetamido)-iminodiacetate (also called ADA). Any counter ion may be used in these salts; ammonium, sodium, and potassium may serve as illustrative examples. Further examples of buffers include, but are not limited to, triethanolamine, diethanolamine, ethylamine, triethylamine, glycine, glycylglycine, histidine, tris(hydroxymethyl)aminomethane (also called TRIS), bis-(2-hydroxyethyl)-imino-tris(hydroxymethyl)methane (also called BIS-TRIS), and N-[Tris(hydroxymethyl)-methyl]-glycine (also called TRICINE), to name a few. The buffers may be aqueous solutions of such buffer compounds or solutions in a suitable polar organic solvent. One or more respective solutions may be used to accommodate the suspected target nucleic acid as well as other matter used, throughout an entire method of the present invention.

Further examples of matter that may be added, include salts, detergents or chelating compounds. As yet a further illustrative example, nuclease inhibitors may need to be added in order to maintain a nucleic acid molecule in an intact state. While it is understood that for the purpose of detection any matter added should not obviate the formation of a complex between the PNA capture molecule (or other nucleic acid capture molecule used in another method of the invention, see below) and the target nucleic acid molecule, for the purpose of carrying out a control measurement a respective agent may be used that blocks said complex formation.

One method of the invention includes immobilizing on the detection electrode a peptide nucleic acid (PNA) capture molecule. As indicated above, a PNA molecule is a nucleic acid molecule in which the backbone is a pseudopeptide rather than a sugar. Accordingly, PNA generally has a charge neutral backbone, in contrast to DNA or RNA. Nevertheless, PNA is capable of hybridising at least complementary and substantially complementary nucleic acid strands, just as e.g. DNA or RNA (to which PNA is considered a structural mimic).

The PNA capture molecule, as well as other nucleic acid capture molecules used in other methods according to the present invention, may be of any suitable length. In some embodiments the PNA capture molecule or other nucleic acid capture molecule has a nucleic acid sequence of a length of about 7 to about 30 bp, for example a length of about 9 to about 25 bp, such as a length of about 10 to about 20 bp.

The PNA capture molecule used in the present invention has a nucleotide sequence that is at least partially complementary to at least a portion of the target nucleic acid molecule. The respective nucleotide sequence of the PNA capture molecule may for example be 70, for example 80 or 85, including 100% complementary to another nucleic acid sequence. The higher the percentage to which the two sequences are complementary to each other (i.e. the lower the number of mismatches), the higher is typically the sensitivity of the method of the invention (see FIG. 7). In typical embodiments the respective nucleotide sequence is substantially complementary to at least a portion of the target nucleic acid molecule. “Substantially complementary” as used herein refers to the fact that a given nucleic acid sequence is at least 90, for instance 95, such as 100% complementary to another nucleic acid sequence. The term “complementary” or “complement” refers to two nucleotides that can form multiple favourable interactions with one another. Such favourable interactions include Watson-Crick base pairing. As an illustrative example, in two given nucleic acid molecules (e.g. DNA molecules) the base adenosine is complementary to thymine, while the base cytosine is complementary to guanine. A nucleotide sequence is the complement of another nucleotide sequence if all of the nucleotides of the first sequence are complementary to all of the nucleotides of the second sequence. Accordingly, the respective nucleotide sequence will specifically hybridise to the respective portion of the target nucleic acid molecule under suitable hybridisation assay conditions, in particular of ionic strength and temperature. Where desired, more than one PNA capture molecule may be immobilized. This may for instance be desired in order to broadly screen for the presence of any of a group of selected target nucleic acid sequences. The use of more than one PNA capture molecule may also be desired for the detection of the same target nucleic acid molecule via different recognition sequences, e.g., the 5′- and 3′-termini thereof, which enhances the likelihood to detect even a few copies of a target nucleic acid molecule in a sample.

In typical embodiments the target nucleic acid molecule includes a pre-defined sequence. In some embodiments the target nucleic acid molecule furthermore includes at least one single-stranded region. In such embodiments it may be desirable to select a single-stranded region as the predefined sequence. In this case the PNA capture molecule can directly form Watson-Crick base pairs with the target nucleic acid molecule, without the requirement of separating complementary strands of the target nucleic acid molecule. Where the target nucleic acid molecule, or a region thereon that includes a predefined sequence, is provided or suspected to be in double strand form, the respective nucleic acid duplex may be separated by any standard technique used in the art, for instance by increasing the temperature (e.g. 95° C., see also the Examples below). In embodiments where multiple sequences may be included in the target nucleic acid molecule, multiple respective PNA capture molecules may be used, each of which being at least partially complementary to e.g. a selected portion of the target nucleic acid molecule (see also below).

The PNA capture molecule may be immobilized on the detection electrode at any stage during the present method of the invention. As two examples, it may be immobilized at the beginning of the method or before adding a polymerisable positively charged precursor (see below). In typical embodiments it is immobilised before performing an electrochemical measurement (see below). The PNA capture molecule may be immobilized by any means. It may be immobilized on the entire surface, or a selected portion of the surface of the detection electrode. In some embodiments the PNA capture molecule is provided first and thereafter immobilized onto the surface of the detection electrode. An illustrative example is the mechanical spotting of the PNA capture molecule onto the surface of the electrode. This spotting may be carried out manually, e.g. by means of a pipette, or automatically, e.g. by means of a micro robot. As an illustrative example, the polypeptide backbone of the PNA capture molecule may be covalently linked to a gold detection electrode via a thio-ether-bond.

The surface of the detection electrode may be activated prior to immobilizing the PNA capture molecule thereon, for instance in order to facilitate the attachment reaction (see also above). Where a glass electrode is used, it may for example be modified with aminophenyl or aminopropyl silanes. 5′-succinylated PNA capture molecules (or in other methods of the invention other nucleic acid capture molecules) may be immobilised thereon by carbodiimide-mediated coupling. In some embodiments the electrode surface may for instance be coated with an electroconductive polymer, such as polypyrrole (Wang, J., et al., Anal. Chem. (1999) 71, 18, 4095-4099; Wang, J., et al., Anal. Chim. Acta (1999) 402, 7-12), polythiophene, polyaniline, polyacetylene, poly(N-vinyl carbazole), or a copolymer such as a copolymer of pyrrole and thiophene or a copolymer of juglone and 5-hydroxy-3-thioacetic-1,4-naphthoquinone (Reisberg, S., et al., Anal. Chem. (2005) 77, 10, 3351-3356). In embodiments where a carbon paste electrode is used, it may for example be modified with carboxyl groups by mixing stearic acid with the paste. A PNA capture molecule may be immobilised on a respective electrode by means of linking molecule ethylenediamine.

As a further illustrative example, a linking moiety such as an affinity tag may be used to immobilize the PNA capture molecule. Examples of an affinity tag include, but are not limited to biotin, dinitrophenol or digoxigenin, oligohistidine, polyhistidine, an immunoglobulin domain, maltose-binding protein, glutathione-S-transferase (GST), calmodulin binding peptide (CBP), FLAG′-peptide, the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the hemagglutinin (HA) epitope of the sequence Tyr-Pro-Tyr-Asp-Val-Pro-Asp-Tyr-Ala, the “myc” epitope of the transcription factor c-myc of the sequence Glu-Gln-Lys-Leu-Ile-Ser-Glu-Glu-Asp-Leu, or an oligonucleotide tag the sequence of which typically differs from the sequence of the target nucleic acid molecule to which a portion of the PNA capture molecule is at least partially complementary. These two nucleotide sequences may differ to such an extent that the sequence of the nucleotide tag is not capable of hybridising to the sequence of any portion of the target nucleic acid molecule. Such an oligonucleotide tag may for instance be used to hybridise to an immobilized oligonucleotide with a complementary sequence. A further example of a linking moiety is an antibody. In respective embodiments an antibody-nucleic acid conjugate may be used as the PNA-capture molecule.

Avidin or streptavidin may for instance be employed to immobilize a biotinylated nucleic acid, or a biotin containing monolayer of gold may be employed (Shumaker-Parry, J. S., et al., Anal. Chem. (2004) 76, 918). As yet another illustrative example, the PNA capture molecule may be locally deposited, e.g. by scanning electrochemical microscopy, for instance via pyrrole-oligonucleotide patterns (e.g. Fortin, E., et al., Electroanalysis (2005) 17, 495). In other embodiments the PNA capture molecule may be directly synthesized on the detection electrode, for example using photoactivation and deactivation.

After immobilizing the PNA capture molecule on the detection electrode, any remaining PNA capture molecule, or molecules, that were not immobilized may be removed from the detection electrode. Removing an unbound PNA capture molecule may be desired to avoid subsequent hybridisation of such PNA molecule with the target nucleic acid molecule, which might reduce the sensitivity of the present method. Removing an unbound PNA capture molecule may also be desired to avoid a non-specific binding of such PNA molecule to any matter present in a sample used, which might for instance alter the conductivity of such matter (e.g., reducible metal cations), which might interfere with the results of the electrochemical measurement (see also below). An unbound capture molecule may for instance be removed by exchanging the medium, e.g. a solution that contacts the detection electrode.

Where desired, a blocking agent may be immobilised on the detection electrode. This blocking agent may serve in reducing or preventing non-specific binding of matter included in the solution expected to include the target nucleic acid molecule. It may also serve in reducing or preventing non-specific binding of any other matter, such as a molecule or solution that is further added to the detection electrode when carrying out the method of the invention.

The blocking agent may be added together with the PNA capture molecule or subsequently thereto. Any agent that can be immobilized on the electrode and that is able to prevent (or at least to significantly reduce) the non-specific interaction between molecules, the detection of which is undesired, and the PNA capture molecule is suitable for that purpose, as long as the specific interaction between the PNA capture molecule and the target nucleic acid molecule is not prevented. Examples of such agents are thiol molecules, disulfides, thiophene derivatives, and polythiophene derivatives. An illustrative example of a useful class of blocking reagents are thiol molecules such as 16-mercaptohexadecanoic acid, 12-mercaptododecanoic, 11-mercaptodecanoic acid or 10-mercaptodecanoic acid.

The term “derivative” as used herein thus refers to a compound which differs from another compound of similar structure by the replacement or substitution of one moiety by another. Respective moieties include, but are not limited to atoms, radicals or functional groups. For example, a hydrogen atom of a compound may be substituted by alkyl, carbonyl, acyl, hydroxyl, or amino functions to produce a derivative of that compound. Respective moieties include for instance also a protective group that may be removed under the selected reaction conditions.

The present method of the invention further includes contacting the electrode with the solution expected to include the target nucleic acid molecule (cf. FIG. 1B, step II). The electrode may for example be immersed in a solution, to which the solution expected to include the target nucleic acid molecule is added. In some embodiments both such solutions are aqueous solutions. In one embodiment the entire method is carried out in an aqueous solution. The method further includes allowing the target nucleic acid molecule to hybridise to the PNA capture molecule on the electrode. As already indicated above, thereby the formation of a complex between the PNA capture molecule and the target nucleic acid molecule is allowed (cf. FIG. 1B, step III). If the solution contains a plurality of different target nucleic acid molecules to be detected, the conditions are chosen so that the target nucleic acid molecules can either bind simultaneously or consecutively to their respective capture molecules.

Where desired, after allowing the target nucleic acid molecule to hybridise to the PNA capture molecule, the solution expected to include the target nucleic acid molecule may be removed. This may for example be desired in order to remove any negatively charged molecules, such as nucleic acid molecules that were present in the solution expected to include the target nucleic acid molecule. Depending on the origin of the respective sample and any extraction used (if any; cf. above) removing components of the respective solution may improve the signal-to-noise ratio of the electrochemical measurement performed in the method of the invention (see below). Repeated replacement of a solution that contacts the detection electrode (“washing”) or rinsing etc. of the electrode may also be performed where desired. Any matter known to be present in a sample used may also be removed by a suitable method that does not dissolve the complex formed between PNA capture molecule and target nucleic acid molecule, such as enzymatically. The same applies for any target nucleic acid molecule that did not hybridise to the PNA capture molecule. As an example, this may be accomplished by an enzyme, which selectively breaks down single-stranded DNA, such as mung bean nuclease, nuclease P1 or nuclease S1.

The method of the present invention also includes providing a polymerisable positively chargeable precursor. The method further includes adding the polymerisable positively chargeable precursor (cf. step IV in FIG. 1B). The polymerisable positively chargeable precursor has an electrostatic net charge that is complementary to the electrostatic net charge of the target nucleic acid molecule. Typically the target nucleic acid molecule is negatively charged in neutral and acidic conditions, in particular where the target nucleic acid molecule is DNA or RNA, due to the backbone of alternating sugar and phosphate molecules in this type of nucleic acid. Accordingly, conditions are selected where the polymerisable positively chargeable precursor has a positive net charge. Those skilled in the art will be aware of the fact that the pK value of a selected precursor molecule serves as a valuable guidance in choosing a suitable pH range or pH value for a solution, in which the precursor molecule is positively charged. This pH range or value is obtained by way of adjusting (e.g. titrating) or by providing the target nucleic acid—and thus typically the detection electrode as well—in a solution of the desired pH. When the polymerisable positively chargeable precursor is added, the target nucleic acid molecule may for example be included in a solution of a pH value selected in the range of about 1.5 to about 8.0, such as in the range of about 1.7 to about 7.0, in the range of about 1.9 to about 6.0, or in the range of about 2.0 to about 5.5. The pH value may thus for instance be selected to be about 3.0 or about 4.0. In some embodiments the pH may also be adjusted to be in a respective range (e.g. of about 1.5 to about 8.0, such as in the range of about 1.7 to about 7.0, in the range of about 1.9 to about 6.0, or in the range of about 2.0 to about 5.5) at the same time as, or after the addition of the polymerisable positively chargeable precursor.

Accordingly, the polymerisable positively chargeable precursor associates to the target nucleic acid molecule, and thus to the complex formed between the PNA capture molecule and the target nucleic acid molecule. Therefore, the polymerisation of the polymerisable precursor can be carried out by means of a suitable reactant molecule. The respective polymerisation will accordingly involve, including start at, a precursor that is associated to the target nucleic acid molecule.

Any positively chargeable precursor may be used as long as it can be polymerised in the presence of the complex between the PNA capture molecule and the target nucleic acid molecule, without dissolving or degrading the respective complex in such a mariner that the target nucleic acid molecule is no longer associated to the detection electrode. As an illustrative example, the polymerisable positively chargeable precursor may be an aromatic amine (see e.g. FIG. 3 for examples), such as aniline, pyridineamine (e.g. 2-pyridineamine, 3-pyridineamine or 4-pyridineamine), pyrrole, imidazole or a derivative thereof. As a further illustrative example, an aniline derivative may also be of the general formula

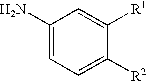

wherein R¹ and R² may independently selected from the group consisting of H, aliphatic, cycloaliphatic, aromatic, arylaliphatic, and arylcycloaliphatic hydrocarbyl groups, comprising 0-5 heteroatoms, i.e. atoms that differ from carbon, for example 0-3 heteroatoms, selected from the group N, O, S, and Si. R¹ and R² may optionally be linked so as to define an aliphatic, cycloaliphatic, aromatic, arylaliphatic, or arylcycloaliphatic hydrocarbyl bridge. In case that a compound is selected that includes a negatively chargeable moiety (or functional group, e.g. carboxyclic, or sulfonic), it may be desired to verify that the respective compound is indeed positively chargeable under the selected conditions. This may for example be done by calculating the respective pK value or by testing whether the compound associates with the selected nucleic acid molecule.

The term “aliphatic” means, unless otherwise stated, a straight or branched hydrocarbon chain, which may be saturated or mono- or polyunsaturated. An unsaturated aliphatic group contains one or more double and/or triple bonds. The branches of the hydrocarbon chain may include linear chains as well as non-aromatic cyclic elements. The hydrocarbon chain, which may, unless otherwise stated, be of any length, and contain any number of branches. Both the main chain as well as the branches may furthermore contain heteroatoms as for instance N, O, S, Se or Si.

The term “alicyclic” means, unless otherwise stated, a nonaromatic cyclic hydrocarbon moiety, which may be saturated or mono- or polyunsaturated. The cyclic hydrocarbon moiety may be substituted with nonaromatic cyclic as well as chain elements. The main chain of the cyclic hydrocarbon moiety may, unless otherwise stated, be of any length and contain any number of non-aromatic cyclic and chain elements. Both the cyclic hydrocarbon moiety and the cyclic and chain substituents may furthermore contain heteroatoms, as for instance N, O, S, Se or Si.

The term “aromatic” means, unless otherwise stated, a planar cyclic hydrocarbon moiety of conjugated double bonds, which may be a single ring or include multiple fused or covalently linked rings. The main chain of the cyclic hydrocarbon moiety may, unless otherwise stated, be of any length and contain any number of heteroatoms, as for instance N, O and S.

By the term “arylaliphatic” is meant a hydrocarbon moiety, in which one or more aryl groups are attached to or are substituents on one or more aliphatic groups. Thus the term “arylaliphatic” includes for instance hydrocarbon moieties, in which two or more aryl groups are connected via one or more aliphatic chain or chains of any length, for instance a methylene group.

Each of the terms “aliphatic”, “alicyclic”, “aromatic” and “arylaliphatic” as used herein is meant to include both substituted and unsubstituted forms of the respective moiety. Substituents my be any functional group, as for example, but not limited to, amino, amido, azido, carbonyl, cyano, isocyano, dithiane, halogen, hydroxyl, nitro, seleno, silyl, silano, thio, thiocyano, and trifluoromethyl.

The present method of the invention further includes providing a suitable reactant molecule that is capable of undergoing a reaction with the positively chargeable precursor that results in polymerisation of the latter. The reactant molecule may also be able to initiate the polymerisation of the polymerisable positively chargeable precursor. As an illustrative example, the reactant molecule may be an oxidant. Examples of a suitable oxidant include, but are not limited to, a ruthenium tris(bipyridinium) complex, a persulfate (such as ammonium persulphate or tetrabutylammonium persulphate), a peroxide (see also below), hydrogen tetrachloroaurate (auric acid, HAuCl₄), a chromate, a dichromate, a manganate, a permanganate, oxygen, ozone, nitrogen oxide, a halogene, a chlorite, a chloride, a perchloride, a chlorate, a iodate, a nitrate, a sulfoxide and osmium tetroxide.

The method of the present invention also includes adding the reactant molecule (cf. step IV in FIG. 1B). The addition of a suitable reactant molecule initiates the polymerisation of the polymerisable positively chargeable precursor (see. FIG. 1B, step V). As an illustrative example, if aniline or a suitable derivative thereof is selected as the respective precursor and an oxidant is selected as the reactant molecule, the polymerisation may start according the following reaction scheme (I) and subsequently continue respectively:

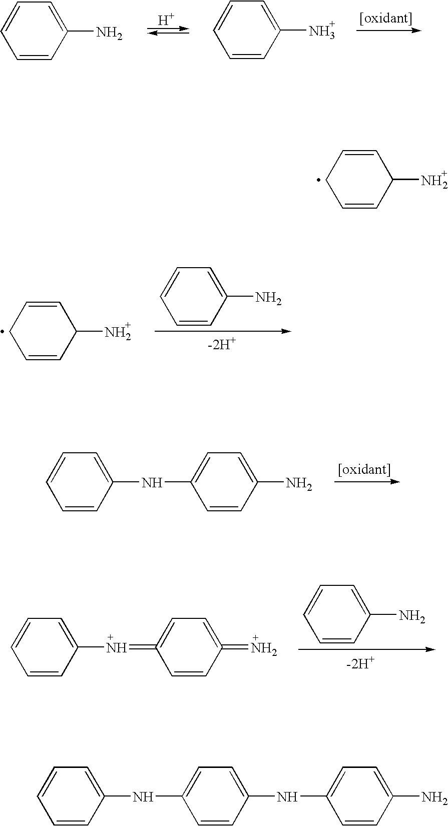

It may also start along the following reaction scheme (II):

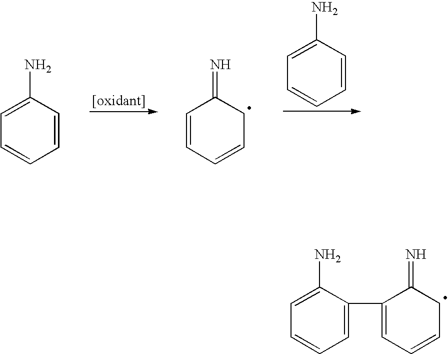

as well as along the following reaction scheme (III):

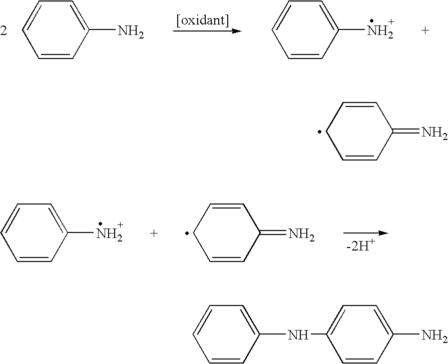

Without the intent of being bound by theory, it is believed that with aniline as the precursor, in the methods of the invention the polymerisation typically occurs at the para position as shown above (scheme I, scheme III). The hybridised anionic target nucleic acid molecule may serve as a template, providing a local environment that guides para-coupling of aniline molecules. Previous studies on enzymatically catalysed polymerisation of aniline have shown that the polymerisation of aniline is catalysed by peroxidases, horse-radish peroxidase for example, under mild conditions (Liu, W., et al., J. Am. Chem. Soc. (1999) 121, 71-78; Nagarajan, R., et al., Macromolecules (2001) 34, 3921-3927; Caramyshev, A. V., et al., Biomacromolecules (2005) 6, 1360-1366.). However, the products of the horse-radish peroxidase-catalysed polymerisation of aniline are a mixture of highly branched ortho- and para-substituted carbon-carbon and carbon-nitrogen polyaniline together with the desired head-to-tail polymerised polyaniline, which greatly diminishes the degree of conjugation and severely affects the electrical, electrochemical, and optical properties of the resulting electroconductive polymers. On the contrary, in the presence of an anionic polyelectrolyte template, a water-soluble, polyaniline/polyelectrolyte complex is formed, in which the polyaniline is wrapped around the polyelectrolyte template (Liu et al., 1999, supra, Nagarajan et al., 2001, supra, Caramyshev et al., 2005, supra). More importantly, polyaniline in the complex retains its full electrochemical activity and is mostly the desired head-to-tail structure with minimal branching, paving the way for the development of ultrasensitive electrochemical nucleic acid biosensors. Later, this enzyme-catalysed template-guided synthetic approach was applied to prepare highly ordered polyaniline (Nagarajan et al., 2001, supra) and polyaniline nanowires on silicon substrates by using fully stretched nucleic acid as growing templates.

As a further illustrative example, if pyrrole or a suitable derivative thereof is selected as the respective precursor and an oxidant is selected as the reactant molecule, the polymerisation may start according the following reaction scheme (IV) and subsequently continue respectively:

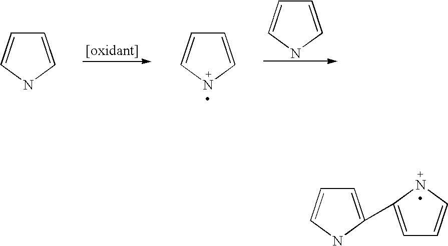

As already mentioned above, the anionic target nucleic acid molecule, which forms a complex with the PNA capture molecule, typically serves as a template for the polymerisation of the positively chargeable precursor. As a consequence, the polymerisation occurs exclusively at the target nucleic acid molecule.

In some embodiments, a suitable initiator may be added. An initiator is generally matter such as a molecule that can generate radical species under mild conditions. Usually it can also promote radical polymerisation reactions. Examples of a respective initiator include, but are not limited to a radical initiator, such as a halogen molecule, an azo compound, a persulfate molecule, a peroxydisulfate ((SO₃)₂O₂²⁻) and a peroxide compound, such as an organic peroxide compound. Two illustrative examples of a halogen molecule are chlorine or bromine. Three illustrative examples of an azo compound are 2,2′-azobis(2-amidinopropane), 2,2′-azobis(2,4-dimethylvaleronitrile) and azobis(isobutyramidine). Two illustrative example of a persulfate molecule are ammonium persulfate and potassium persulfate. Where desired, a persulfate molecule may also be combined with a thiosulfate (e.g. sodium thiosulfate, Na₂S₂O₃), a bisulfite, a dithionite, or an ascorbic acid molecule. Examples of an organic peroxide compound include, but are not limited to, benzoyl peroxide, cumenyl peroxide, dicumyl peroxide, cumene hydroperoxide, diphenyl peroxide, bis-(tert-butyl)peroxide, bis(tert-butylperoxysuccinyl) peroxide, a lipid peroxide such as lauroyl peroxide, diacetyl peroxide or trifluoroacetyl peroxide, bis(o-iodophenylacetyl) peroxide, ethyl peroxide, 2-naphthyl peroxide, 2-naphthoyl peroxide, butyl hydroperoxide, tert-butyl hydroperoxide, vinyl hydroperoxide, cyclohexyl hydroperoxide, trifluoromethyl peroxide, 2,5-bis(1-methylethyl)phenyl-hydroperoxide, 1,5-dimethyl-6,8-bis(trimethylsilyl)-2,3-dioxabi-cyclo[2.2.2]oct-7-en-5-yl hydroperoxide, and p-1,1-dimethylethyl-1-methyl-3-phenyl-2-propynyl peroxide. A photoinitiator such as dibenzyl ketone, camphorquinone, 1-(bromoacetyl)pyrene, α-ketoglutaric acid, or an uranyl salt may likewise be employed.

Where desired, for example to accelerate the polymerisation, or in embodiments where the reactant molecule undergoes a sluggish reaction, a suitable catalyst may furthermore be provided and the reactant molecule be exposed thereto. Examples of a catalyst include, but are not limited to, light, a metal halide such as a metal chloride or a metal bromide, a metal sulphate, and an enzyme or an enzyme-conjugate. Examples of suitable metal halides include, but are not limited to a ferrous halide, e.g. ferrous chloride, a lithium halide, e.g. lithium chloride, a copper halide, e.g. a copper(I) halide or a copper(II) halide such as CuCl₂ or CuBr₂, a molybdenum halide such as MoCl₅, an iridium halide, such as (NH₄)₂IrCl₆, a manganese chloride such as MnCl₂, a nickel halide such as NiCl₂, a titanium chloride such as titanium trichloride (TiCl₃), and an aluminium halide, such as aluminium chloride. Two examples of a suitable metal sulphate FeSO₄ and CuSO₄. Where the catalyst is e.g. a compound or a powder, a respective catalyst may for example be added to the detection electrode, on which the PNA capture molecule is immobilised. It may, for instance, be added after the polymerisable positively chargeable precursor has associated to the complex formed between the PNA capture molecule and the target nucleic acid molecule. It may for instance also be added together with the reactant molecule or subsequently thereto. The catalyst may for instance be selected in such a way that the reactant molecule is a substrate for the catalyst.

In embodiments where an enzyme or an enzyme-conjugate is used as a catalyst, any enzyme may be used that is capable of catalysing the polymerisation of the positively chargeable precursor. The enzyme may for instance be selected in such a way that the reactant molecule is a substrate for the enzyme. As an illustrative example of an enzyme suitable for the purposes of the present invention, an oxidoreductase enzyme may be selected. Examples of an oxidoreductase enzyme include, but are not limited to, a peroxidase, an oxidase, a dehydrogenase, a monooxygenase, a hydroxylase, a dioxygenase, or a hydrogenase.

A respective peroxidase enzyme may for instance be a haem peroxidase enzyme. Examples of a haem peroxidase enzyme include, but are not limited to, horseradish peroxidase, cytochrome c peroxidase, glutathione peroxidase, myeloperoxidase, thyroid peroxidase, eosinophil peroxidase, lactoperoxidase, ascorbate peroxidase, peroxidasin, prostaglandin H synthase, a bacterial catalase-peroxidase such as E. coli catalase-peroxidase, M. tuberculosis catalase-peroxidase, or Bacteroides fragilis catalase-peroxidase, lignin peroxidase, plant ascorbate peroxidase, Haem chloroperoxidase, manganese peroxidase, stigma specific peroxidase, Euphorbia characias latex peroxidase, Arthromyces ramosus peroxidase, sorghum grain peroxidase SPC4, soybean peroxidase, Phanerochaete chrysosporium manganese-dependent peroxidase, lacrimal gland peroxidase, or any combination thereof. Examples of an oxidase enzyme include, but are not limited to, the oxygen oxidase laccase, glucose oxidase, lactase oxidase, pyruvate oxidase, tyrosinase or any combination thereof. A respective peroxidase enzyme may also be a non-haem peroxidase enzyme such as for instance bromoperoxidases BPO-A1, BPO-A2 or BPO-B, the non-haem chloroperoxidase from Pseudomonas fluorescens, or the non-haem extracellular peroxidase from Thermomonospora fusca BD25. A secreted mixture or an extract of enzymes may also be used as a catalyst where desired. As an illustrative example, the so-called “white-rot fungi” produces lignin-modifying extracellular enzymes (LME) that include two peroxidase enzymes (lignin peroxidase and Mn peroxidase), an oxidase enzyme laccase and an oxidase enzyme aryl alcohol oxidase. A respective crude extract may be used as a catalyst in a method of the present invention (cf. also Curvetto, N. R., et al., Biochemical Engineering Journal (2006) 29, 3, 191-203). Further examples of suitable catalysts include haematin (Curvetto et al., 2006, supra) and haemoglobin.

In one embodiment the oxidant is a peroxide such as hydrogen peroxide, and the catalyst is horse radish peroxidase. Horse radish peroxidase has previously been reported (Liu, W., et al., J. Am. Chem. Soc. (1999) 121, 71-78; Nagarajan, R., et al., Macromolecules (2001) 34, 3921-3927) to catalyse the oxidative polymerisation of aniline in the presence of the oxidant hydrogen peroxide (H₂O₂). This polymerisation can also be carried out in the presence of DNA, which expedites the polymerisation reaction (Nickels, P. et al., Nanotechnology (2004) 15, 1524-1529). In another embodiment the oxidant is molecular oxygen (O₂) and the catalyst is laccase (e.g. isolated from Coriolus hirsutus). Laccase C. hirsutus has been reported to possess a higher operational stability than horse radish peroxidase under acidic conditions in the polymerisation of aniline (Karamyshev, A. V. et al., Enzyme and Microbial Technology (2003) 33, 5, 556-564).

In some embodiments the catalyst is in solution (see FIG. 8) or in suspension. In some embodiments the catalyst is coupled to a detection probe. The detection probe may for example be a label that emits light of a certain wavelength upon irradiation (e.g. fluorescein or a fluorescent protein), thus enabling an optical control measurement to verify the presence of the catalyst. The detection probe may also be a detection probe nucleic acid molecule. In some embodiments the catalyst is an enzyme or an enzyme-conjugate to which a detection probe nucleic acid molecule is coupled. The detection probe nucleic acid molecule may be any nucleic acid of any length, for example of about 5 to about 250 bp, such as about 5 to about 100 bp. Such a nucleic acid molecule is at least partially complementary to at least a portion of the target nucleic acid molecule. The detection probe, which is attached to the catalyst, hybridises to a respective portion of the target nucleic acid different from the portion to which the capture nucleic acid molecule hybridises. Accordingly, the detection probe directs the catalyst to the target nucleic acid and associates it thereto.

Where desired, further methods for detection may be employed. As an example, an optical detection may also be performed or enhanced by means of an optically amplifying conjugated polymer, e.g. in a Förster energy transfer system (Gaylord, B. S., et al., Proc. Natl. Acad. Sci. USA (2005) 102, 34-39; Gaylord, B. S., et al., J. Am. Chem. Soc. (2003) 125, 896-900). As a further example, a cationic polythiophene may be added, which changes its color and fluorescence in the presence of single-stranded or double-stranded nucleic acid molecules (Ho, H. A., et al., J. Am. Chem. Soc. (2005) 127, 36, 12673-12676).

The polymerisation of the polymerisable positively chargeable precursor forms an electroconductive polymer. As already explained above, due to the (electrostatic) association of the polymerisable positively chargeable precursor to the complex formed between the PNA capture molecule and the target nucleic acid molecule, this electroconductive polymer is likewise associated with the complex formed between the PNA capture molecule and the target nucleic acid molecule. The present inventors have found that the electroconductive polymer is usually robustly bound to the detection electrode. For example extensive washing and potential cycling thereafter produced no noticeable changes in subsequent electrochemical measurements. Where desired, further reactions, washing steps and additional detection methods etc. may therefore be performed. The electroactivity of the electroconductive polymer allows ultrasensitive electrochemical detection and quantification of target nucleic acid molecules as explained in the following.

The electroconductive polymer formed allows the transfer of electrons to and from the detection electrode. The electroconductive polymer may for example include moieties such as functional groups (e.g. amino groups) that are capable of accepting and/or donating electron density, thus allowing the flow of electrons from/to the detection electrode (see FIG. 2A and FIG. 2B for illustrative examples). While the electroconductive polymer is already partially formed and the polymerisation reaction still continues, electrons may for instance also be transferred from the electrode via the electroconductive polymer to the oxidant during a polymerisation reaction as illustrated by examples in schemes (I) to (IV) and FIG. 2C. As depicted in these examples, electrons removed from the growing polymer chain by the oxidant may for instance be replaced by electrons from the detection electrode.

The present invention thus uses the dynamic growing process of an electroconductive polymer as a means to both generate and amplify an electrochemical signal. A longer electroconductive polymer generally includes more moieties, e.g. functional groups such as amino groups that are capable of accepting and/or donating electron density. Accordingly, a longer polymer generally has a higher capacity of allowing the flow of electrons from/to the detection electrode (cf. e.g. FIG. 2A and FIG. 2B). As mentioned above (see also the discussion in the example below), the obtained polymer will typically furthermore include branches of various degree, rather than form a purely linear product. Such branches also amplify signals, including signals from the respective growing ends of the polymer (cf. e.g. FIG. 2C). Furthermore typically more than one polymerisable positively chargeable precursor associates to one target nucleic acid molecule, such that multiple polymer chains start at a single target nucleic acid molecule. The present invention thus includes a highly efficient signal amplification and transduction route.

Because the polymer associated to the target nucleic acid is electroactive, the current generated from it nevertheless directly correlates to the concentration of nucleic acid in the sample solution. The combination of highly efficient polymerization, guided-deposition, and electrochemical detection (see below) thus provides a generic platform for ultrasensitive detection of nucleic acids. Furthermore, the catalytic and cumulative nature of the system causes the signal to increase with increasing incubation time before sampling. Essentially the signal is only limited by capacity of the selected system in terms of the amounts of reactant molecule and polymerisable precursor (see FIG. 5), and where applicable, the amount of catalyst in the system. As a result, a longer incubation period produces a higher signal and a lower detection limit (see e.g. FIG. 5D). In an example described below, an incubation period of 30 minutes was found to result in a detection of as little as 2 fM target nucleic acid molecule and a linear current-concentration relationship up to 2 pM.

Accordingly, the occurrence of a polymerisation can be detected by way of an electrochemical measurement. The present method of the invention thus also includes performing an electrochemical measurement at the detection electrode (see step VI in FIG. 1B). An electrochemical measurement according to the invention may include a measurement of current as well as of voltage. Any electrochemical technique may be used in the present or any other method of the present invention. Examples include, but are not limited to, linear cyclic voltammetry, square wave voltammetry, normal pulse voltammetry, differential pulse voltammetry and alternating current voltammetry. The electrochemical measurement may be oxidative or reductive. Two illustrative examples of electrochemical measurements according to the present method of the invention are shown in FIGS. 8 and 9.

In typical embodiments, the result obtained is then compared to that of a control measurement. In a respective control measurement PNA capture molecules unable to bind the target nucleic acid to be detected may for instance be used. An example of such a “control” PNA capture molecule is a PNA molecule having a sequence not complementary to any portion of the target nucleic acid molecule (see e.g. FIG. 8 and FIG. 9). If the two electrical measurements, i.e. “sample” and “control” measurement, differ in such a way that the difference between the values determined is greater than a pre-defined threshold value, the sample solution contained the relevant target nucleic acid molecule.

In some embodiments, the method is designed in such a way that a reference measurement and a measurement for detecting a target nucleic acid molecule are performed simultaneously. This may for instance be done by carrying out a reference measurement only with a control medium and, at the same time, a measurement with the sample solution expected to contain the target nucleic acid to be detected. Likewise, a respective control measurement with a PNA capture molecule that is not complementary to any portion of the target nucleic acid molecule may be carried out in parallel to a measurement for detecting a target nucleic acid molecule.

The present method also allows detecting more than one target nucleic acid molecule simultaneously or consecutively in a single measurement. For this purpose, a substrate including a plurality of detection electrodes as described above may for example be used, wherein different types of PNA capture molecules, each of which exhibiting (specific) is capable of hybridising to at least a portion of a particular target nucleic acid molecule, are immobilized on the detection electrodes. Alternatively, it may also be possible to use a plurality of detection electrodes, each of which being provided with only one type of PNA capture molecule.

The method according to the invention may be carried out by using virtually any electrode arrangement known in the art that includes a detecting or working electrode. Such an electrode arrangement usually also include a counter electrode as well as a reference electrode. The detecting electrode may be a conventional metal electrode (gold electrode, silver electrode etc.) or an electrode made from polymeric material or carbon. An electrode arrangement that comprise the detecting electrode may also be a common silicon or gallium arsenide substrate, to which a gold layer and a silicon nitride layer have been applied, and which has subsequently been structured by means of conventional lithographic and etching techniques to generate the electrode arrangement(s).

The method according to the present invention also allows detecting more than one type of analyte simultaneously or consecutively in a single measurement. For this purpose, a substrate comprising a plurality of electrode arrangements as disclosed herein may be used, wherein different types of capture molecules, each of which exhibiting (specific) binding affinity for a particular analyte to be detected, are immobilized on the electrodes of the individual electrode arrangements. Alternatively, it may also be possible to use a plurality of electrode arrangements, each of which being provided with only one type of capture molecules.

An example of an electrode arrangement, which may be used for carrying out the present method, as well as any other method according to the invention, is a conventional interdigitated electrode. Consequently, an arrangement provided with a plurality of interdigitated electrodes, i.e. an electrode array, can be employed for parallel or multiple determinations. Another usable electrode arrangement is an electrode arrangement in the form of a trench or a cavity, which is formed, for example, by holding regions such as, for example, a gold layer on which the capture molecules capable of binding the analytes are immobilized being located on two opposite side walls.

Another method of the invention includes immobilizing on the detection electrode a nucleic acid capture molecule that has a nucleotide sequence that is at least partially to at least a portion of the target nucleic acid molecule. This capture molecule may be any nucleic acid as long as it is capable of hybridising to at least a portion of the sequence of the target nucleic acid molecule. The capture molecule may for example be a DNA, RNA or PNA molecule. A respective nucleic acid molecule may be immobilized by any means, as long as it can hybridise to the target nucleic acid molecule thereafter. Examples of immobilising a nucleic acid capture molecule have already been explained above. As a further example, in embodiments where gold detection electrode is used, an ionisable thiol compound such as 2-dimethylaminoethanethiol hydrochloride may be covalently linked to the surface of the detection electrode. This modification allows nucleic acid molecules to bind through electrostatic interactions.

Where desired, more than one nucleic acid capture molecule may be immobilized on the electrode. This may for instance be desired in order to broadly screen for the presence of any of a group of selected nucleic acid sequences. This may also be desired to allow for the simultaneous or consecutive detection of different analytes such as two or more genomic DNAs, each of them having binding specificity for one particular type of capture molecule. In some embodiments similar nucleic acid sequences, e.g. a number of nucleic acid sequences that are partially or substantially complementary to a selected target nucleic acid molecule, may be immobilized in order to enhance the likelihood of detecting the respective target nucleic acid molecule. Where desired, a further selectivity may be introduced by the selection of the nucleic acid molecule used that is attached to the enzyme added (see below). Furthermore, in this manner the detection of the same target nucleic acid molecule via different recognition sequences can be achieved, e.g., the 5′- and 3′-termini of a nucleic acid molecule, which enhances the likelihood to detect even a few copies of a target nucleic acid molecule in a sample.

It is understood that the above explanations with respect to a PNA capture molecule respectively also apply to a nucleic acid used in the present method of the invention. In the light of the detailed explanations of the previous method of the invention, explanations with regard to the present method of the invention are focussed on differences between these two methods.

The present method also includes contacting the detection electrode with a solution expected to include the target nucleic acid molecule (see FIG. 1A, step II), and allowing the target nucleic acid molecule to hybridise to the nucleic acid capture molecule on the electrode (see FIG. 1A, step III), thereby allowing the formation of a complex between the nucleic acid capture molecule and the target nucleic acid molecule (cf. above).

Similar to the first method described above, the present method also includes adding a polymerisable positively chargeable precursor (see FIG. 1A, step VI). As this polymerisable positively chargeable precursor has an electrostatic net charge that is complementary to the electrostatic net charge of the target nucleic acid molecule, it associates with the complex formed between the nucleic acid capture molecule and the target nucleic acid molecule (see above).

The present method further includes adding a suitable substrate molecule (see FIG. 1A, step VI). Any substrate molecule may be used that is suitable as a substrate for the enzyme used (see above for examples) in the present method. As an illustrative example, the enzyme may be a peroxidase enzyme, for instance a haem peroxidase. In such embodiments the substrate will typically be a peroxide. Any peroxide that can, at least to a certain degree, be dissolved in a selected solution used in this method or the method described above, such as an aqueous solution, may be used in the present invention. Examples of a suitable peroxide include, but are not limited to, hydrogen peroxide, nitrogen peroxide, magnesium peroxide, calcium peroxide, zinc peroxide, benzoyl peroxide, cumenyl peroxide, dicumyl peroxide, diphenyl peroxide, bis(tert-butylperoxysuccinyl) peroxide, a lipid peroxide such as lauroyl peroxide, diacetyl peroxide or trifluoroacetyl peroxide, bis(o-iodophenylacetyl) peroxide, ethyl peroxide, 2-naphthyl peroxide, 2-naphthoyl peroxide, butyl hydroperoxide, vinyl hydroperoxide, cyclohexyl hydroperoxide, trifluoromethyl peroxide, 2,5-bis(1-methyl-ethyl)phenyl-hydroperoxide, 1,5-dimethyl-6,8-bis(trimethylsilyl)-2,3-dioxabicyclo[2.2.2]oct-7-en-5-yl hydroperoxide, and p-1,1-dimethylethyl-1-methyl-3-phenyl-2-propynyl peroxide. As explained above, in some embodiments the enzyme may also be an oxidase enzyme such as for instance laccase. In this case oxygen may for instance conveniently be added together with any aqueous solution used in the method of the invention, in which it is dissolved. In such embodiments, no additional measures need to be taken to add oxygen. Where desired, this may however be carried out by mechanical action, such as stirring, rotating or shaking.

The enzyme, the addition of which is also included in the present method (see FIG. 1A, step IV), is attached to a detection probe nucleic acid molecule. Any nucleic acid molecule may be used for this purpose that is at least partially complementary to at least a portion of the target nucleic acid molecule. Accordingly, the detection probe hybridises to a respective portion of the target nucleic acid different from the portion to which the capture nucleic acid molecule hybridises (cf. also above).

As indicated above, the catalyst, such as an enzyme used in the method described above, may optionally also be attached to a detection probe nucleic acid molecule. Where in the present method of the invention a DNA or RNA molecule are selected as the nucleic acid capture molecule, both the respective nucleic acid capture molecule and the target nucleic acid molecule may however be negatively charged under the assay conditions selected. As explained above, upon addition of the polymerisable positively chargeable precursor the target nucleic acid molecule may for instance be included in a solution of a pH value selected in the range of about 1.5 to about 8.0, such as in the range of about 1.7 to about 7.0, in the range of about 1.9 to about 6.0, or in the range of about 2.0 to about 5.5 (including a value of about 3.0 or about 4.0). The pH may also be brought to a desired range or value, such as one of the aforementioned ranges, during or after adding the polymerisable positively chargeable precursor. In any such embodiments various DNA and RNA molecules will typically have a negatively charged backbone. Accordingly, the positively chargeable precursor may bind to either nucleic acid molecule. Additional selectivity is however introduced into the present method by the use of an enzyme, which is attached to a detection probe nucleic acid molecule. As can be inferred from FIG. 4, the enzyme selectively locates at the target nucleic acid molecule.

The present method also includes allowing the detection probe nucleic acid molecule to hybridise to the target nucleic acid molecule (see FIG. 1A, step V). As already explained above, the present method also includes catalysing the polymerisation of the polymerisable positively chargeable precursor. Since the enzyme, which catalyses the polymerisation, is associated with the complex formed between the nucleic acid capture molecule and the target nucleic acid molecule (via the detection probe nucleic acid molecule), the polymerisation usually starts at the target nucleic acid molecule. At this location the polymerisation accordingly also progresses. The target nucleic acid/enzyme adduct accordingly catalyses the polymerisation of the precursor and guides the deposition of electroconductive polymer at the detection electrode. The electroconductive polymer, which is formed from the polymerisable precursor, is thus associated with the complex formed between the nucleic acid capture molecule and the target nucleic acid molecule.

As for the method described above, the present method further includes performing an electrical measurement at the detection electrode, and detecting and/or quantifying the presence of the target nucleic acid molecule (supra). An illustrative example of an electrochemical measurement according to the present method of the invention is for instance shown in FIG. 4B.

The methods according to the present invention may be a diagnostic method for the detection and/or quantification of one or more target genes. The target gene may be involved in or associated with a disease or a state of the human or animal body that requires prophylaxis or treatment.

The method of the invention may be combined with other analytical and preparative methods. As already indicated above, the target nucleic acid may in some embodiments for instance be extracted from matter in which it is included. Examples of other methods that may be combined with a method of the present invention include, but are not limited to isoelectric focusing, chromatography methods, electrochromatographic, electrokinetic chromatography and electrophoretic methods. Examples of electrophoretic methods are for instance free flow electrophoresis (FFE), polyacrylamide gel electrophoresis (PAGE), capillary zone or capillary gel electrophoresis. Furthermore the data obtained using the present invention may be used to interact with other methods or devices, for instance to start a signal such as an alarm signal, or to initiate or trigger a further device or method.

The present invention also provides kits for the electrochemical detection of a target nucleic acid molecule, which may for instance be diagnostic kits. A respective kit includes a detection electrode. According to one embodiment, the kit also includes a PNA capture molecule, which has a nucleotide sequence that is at least partially complementary to at least a portion of the target nucleic acid molecule (see above). The kit also includes a polymerisable positively chargeable precursor. Illustrative examples of suitable precursors have been given above and are also found in FIG. 3. As explained above, the electrostatic net charge of the polymerisable positively chargeable precursor is complementary to the electrostatic net charge of the target nucleic acid molecule. The kit also includes a suitable reactant molecule, such as an oxidant. In some embodiments the kit may also include a suitable initiator, for example a halogen molecule, an azo compound, a persulfate molecule, and a peroxide compound. Furthermore, in some embodiments the kit may include a catalyst, e.g. a metal chloride, a metal bromide, a metal sulphate or an enzyme (e.g. a peroxidase enzyme or an oxidase enzyme). The catalyst may for example be in solution or coupled to a detection probe nucleic acid molecule. As explained above, in embodiments where the kit includes a catalyst, the reactant may be a substrate molecule for the catalyst.

A respective kit may furthermore include means for immobilizing the capture molecule to the surface of the detection electrode. As explained above, a nucleic acid capture molecule included in the kit may have a moiety that allows for, or facilitates, an immobilisation on a respective detection electrode. The kit may also include a linking molecule. As an illustrative example, 6-mercapto-1-hexanol may be included in the kit. Upon using a respective kit, the PNA capture molecule may then be 5′-C₆H₁₂SH-modified (see above for examples).

According to another embodiment, a kit also includes a nucleic acid capture molecule, which has a nucleotide sequence that is at least partially complementary to at least a portion of the target nucleic acid molecule (see above). The kit further includes a polymerisable positively chargeable precursor, e.g. an aromatic amine. As explained above, the electrostatic net charge of the polymerisable positively chargeable precursor is complementary to the electrostatic net charge of the target nucleic acid molecule. The kit also includes a substrate molecule, such as a peroxide or oxygen dissolved in a solution. As indicated above, oxygen dissolves in aqueous solutions, so that any aqueous solution included in the kit may be the source of this substrate molecule. Furthermore, the kit includes an enzyme attached to a probe nucleic acid molecule (see above for examples). The probe nucleic acid molecule is at least partially complementary to at least a portion of the target nucleic acid molecule. A respective kit may furthermore include means for immobilizing the capture molecule to the surface of the detection electrode as described above.

A respective kit may be used to carry out a method according to the present invention. It may include one or more devices for accommodating the above components before, while carrying out a method of the invention, and thereafter. As an illustrative example, it may include a microelectromedical system (MEMS).

The present invention is of particular significance in the design and manufacture of ultrasensitive non-labelling nucleic acid biosensors and biosensor arrays. The nucleic acid-guided deposition of an electroconductive polymer such as polyaniline combined with efficient biocatalysis offers a very attractive alternative to hybridisation-based nucleic acid biosensors. The skilled artisan will further appreciate that the present invention also allows for the fabrication of simple, low-cost, and portable electrochemical nucleic acid detection devices, providing fast, cheap and simple solutions for e.g. molecular diagnosis, particularly for cancer diagnosis, point-of-care, and field uses. For instance, point-of-care applications require systems that are portable, robust and easy to use, coupled with a reliable assay. Electrochemical systems meet and/or exceed all these requirements. Furthermore, this unique combination of amplification by way of polymerisation and template-guided deposition can be used in conjunction with other detection techniques such as quartz-crystal microbalance, surface plasmon resonance, fluorometry, and colorimetry.

In order that the invention may be readily understood and put into practical effect, particular embodiments will now be described by way of the following non-limiting examples.

### Chemicals

Aniline and 4-aminothiophenol were selected as model compounds. They were obtained (Aniline 99.5%, 4-aminothiophenol 97%) from Sigma-Aldrich (St Louis, Mo.). Thiolated peptide nucleic acid probes used in the following example were custom-made by Eurogentec (Herstal, Belgium). Horse-radish peroxidase labelled detection probes were purchased form Invitrogen (Carlsbad, Calif.) and all other oligonucleotides of PCR purity were from 1st Base Pte Ltd (Singapore). All other reagents of certified analytical grade were obtained from Sigma-Aldrich and used without further purification. A buffer solution of 10 mM Tris-HCl, pH 8.5, 1.0 mM EDTA, and 0.10 M NaCl was used as the hybridisation and washing buffer. A 0.10 M phosphate buffer of pH 4.0 was used as the deposition buffer for polyaniline and the supporting electrolyte.

### Apparatus

Electrochemical experiments were carried out using a CH Instruments model 760b electrochemical workstation coupled with a low current module (CH Instruments, Austin, Tex.). A conventional three-electrode system, consisting of a 3.0-mm-diameter gold working electrode, a nonleak miniature Ag/AgCl reference electrode (Cypress Systems, Lawrence, Kans.), and a platinum wire counter electrode, was used in all electrochemical measurements. To avoid the spreading of the sample droplet beyond the 3.0-mm diameter working area, a patterned hydrophobic film was applied to the gold electrode after immobilising the nucleic acid capture molecule. The electrochemical techniques used in the following are cyclic voltammetry and square wave voltammetry (SWV). SWV experiments were conducted with a pulse amplitude of 25 mV, a step of 3 mV, and a frequency of 15 Hz. All potentials given herein were referred to the Ag/AgCl electrode.

### Biosensor Preparation, Hybridization, and Detection

The pre-treatment of the gold electrode and the immobilization of the nucleic acid capture molecule thereon were carried out as previously described (Xie, H., et al., Anal. Chem. (2004) 76, 1611-1617). Optically polished p-type single-crystal silicon wafer (111) was thoroughly cleaned sequentially in absolute ethanol, 0.50 mol/L NaOH-10% H₂O₂ and boiling water. Gold film (250-nm thick) was deposited in a SEC 1000-RAP electron beam evaporator (CHA Industries, Fremont, Calif.). Gold (99.99%) was evaporated from a tungsten boat at a pressure lower than 2×10⁻⁶ Ton, and a deposition rate of 0.10 nm/s. Following gold deposition, the silicon wafer was annealed at 250° C. in air for 3 h. Prior to nucleic acid capture molecule immobilization, the gold electrode was cleaned in an oxygen plasma (2.5 mA, 0.10 mbar for 2 min) and then immersed in 50 ml of absolute ethanol for 20 min. Three pairs of nucleic acid capture molecules were immobilised in separate experiments:

| | |
|---|---|
| GAPDH capture molecules: | |
| 5′-TTACTCCTTGGAGGCCATGT-3′, | (SEQ ID NO: 1) |
| and | |
| 5′-ATGGTGAAGGTCGGTGTCAA-3′. | (SEQ ID NO: 2) |
| BRCA1 capture molecules: | |
| 5′-TTGGGAGGGGGCTCGGGCAT-3′, | (SEQ ID NO: 3) |
| and | |
| 5′-CAGAGCTGGCAGCGGACGGT-3′. | (SEQ ID NO: 4) |
| Histone H4 capture molecules: | |
| 5′-AATCCGCGATGCAGTTACCT-3′, | (SEQ ID NO: 5) |
| and | |
| 5′-AGTGTCGGTACCTGCACC-3′. | (SEQ ID NO: 6) |

The respective nucleic acid capture molecule was adsorbed onto the gold electrode by immersing it in a solution of phosphate buffered saline (PBS) containing 100 g/ml nucleic acid capture molecule.

After adsorption, the electrode was copiously rinsed with PBS and soaked in stirred PBS for 20 min, rinsed again, and blown dry with a stream of air, a procedure aimed at removing any nonspecifically adsorbed materials. All electrodes coated with nucleic acid capture molecules were characterized by contact angle goniometry and voltammetry. These routine tests ensure that only good coatings with nucleic acid capture molecule were selected for subsequent studies. The gold electrode coated with the nucleic acid capture molecules was immersed into an ethanolic solution of 5.0 mg/ml 16-mercaptohexadecanoic acid (90%, Sigma-Aldrich, St. Louis, Mo.) for 6-12 h to improve the quality and stability. The electrode was then thoroughly washed by immersing in 50 ml of ethanol under vigorous stirring for 10 min, followed by thorough rinsing with ethanol and water. The electrode was ready after air-drying. The surface density of the immobilized nucleic acid capture molecules was =2.30×10⁻¹¹ mol/cm².

The hybridisation of the target nucleic acid and its electrochemical detection were carried out in three steps, as depicted in FIG. 1. In a first method (FIG. 1A), the detection electrode was first placed in a moisture saturated environmental chamber maintained at 50° C. An aliquot of hybridisation solution of 2.5-ml, containing the target nucleic acid, was uniformly spread onto the detection electrode. It was then rinsed thoroughly with a blank hybridisation buffer at 50° C. after a 60-min hybridisation period, and further hybridised at 25° C. for 30 min with a 5.0-ml aliquot of 100 mg/ml of horse-radish peroxidase labelled oligonucleotide detection probes in the hybridisation buffer. Finally, after a thorough rinsing with a pH 9.5 hybridisation buffer the biosensor was incubated in a mixture of aniline/H₂O₂ (30 mM/2.5 mM) in the 0.10 M phosphate buffer of pH 4.0 for 30 min. The polyaniline electrooxidation current in SWV was measured in the 0.10 M phosphate buffer of pH 4.0. In the case of lower nucleic acid concentrations, smoothing was applied after each measurement to remove random noise and electromagnetic interference.

A second method (FIG. 1B) differed from the outlined scheme in that (a) the second hybridisation (25° C. for 30 min with a horse-radish peroxidase labelled oligonucleotide detection probe) was omitted and (b) either horse-radish peroxidase (i.e. unlabelled) was used as a catalyst, or that ammonium persulfate was added. In the latter case, incubation was carried out in the pH 4.0, 0.10 M phosphate buffer containing 2 mM NH₄S₂O₈, 30 mM aniline/2.5 mM H₂O₂. GAPDH capture molecules of SEQ ID NO: 1 and SEQ ID NO: 2 (see above) were used for these measurements.

### Detection Scheme

FIG. 1A shows step-by-step of the working principle of the first method used in this example. A mixed monolayer of thiolated PNA capture molecules and 4-aminothiophenol was self-assembled on the gold electrode, acting as the bioaffinitive sensing interface (I). The interaction of the PNA capture molecule with the target nucleic acid molecule (II) forms a heteroduplex, bringing a high density of negative charges on the biosensor surface (III). Further hybridisation with the horse-radish peroxidase labelled detection probe oligonucleotide (IV) resulted in the formation of a sandwich structure, immobilizing horse-radish peroxidase on the surface of the detection electrode (V). In the second method used in this example (FIG. 1B), the catalyst horse-radish peroxidase was not attached to a detection probe oligonucleotide, so that this further hybridisation could be omitted (cf. FIG. 1B). The same applied for the addition of ammonium persulfate.

Thereafter a mixture of aniline/H₂O₂ in the 0.10 M phosphate buffer of pH 4.0 was added (VI). The hybridised anionic nucleic acid molecule served as a template, providing the requisite local environment to facilitate para-coupling of aniline molecules, and horse-radish peroxidase was the catalyst/initiator. As a result, deposition of polyaniline occurred exclusively at the hybridised target nucleic acid molecule (VII). The electroactivity of the deposited polyaniline allowed for an ultrasensitive electrochemical quantification of the target nucleic acid molecule (VIII). While any nucleic acid molecule may be used, in the present example PNA was selected as the nucleic acid capture molecule for two reasons, namely (a) to minimize non-hybridisation-related uptake of horse-radish peroxidase and (b) to increase the hybridisation efficiency. The neutral character of the PNA backbone alleviates electrostatic interaction between surface immobilized nucleic acid capture molecule and cationic horse-radish peroxidase, and the electrostatic repulsion of duplex formation (Egholm, M., et al., Nature [1993] 365, 556-568; Giesen, U., et al., Nucleic Acids Res. [1998] 26, 5004-5006), producing a high signal/noise ratio. In addition, the mismatch discrimination of PNA is in many cases significantly better than that of DNA (ibid, and Holmen, A., et al., Biochemistry (2000) 39, 7781-7791) offering a higher specificity.

### Feasibility Study of Nucleic Acid Detection

In a preliminary hybridisation test, a mixture of PCR-amplified genes (cDNAs) containing a housekeeping gene, glyceraldehyde-3-phosphate dehydrogenase (GAPDH; 1008 bp), was used as sample target nucleic acid without further purification. Prior to hybridisation, GAPDH was denatured at 95° C. for 5 min to separate DNA duplex. PNA capture molecules complementary to GAPDH were used (see above). Upon hybridisation at 50° C., target GAPDH in the mixture was selectively bound by its complementary nucleic acid capture molecule and immobilized on the biosensor surface. Catalyst labels (horse-radish peroxidase) were brought to the surface of the detection electrode via a second hybridisation with the horse-radish peroxidase-labelled detection probe nucleic acid molecules. After a 30-min incubation period in the mixture of aniline/H₂O₂, the obtained biosensor was examined voltammetrically and compared to a gold electrode coated with a conventional electrodeposited thin polyaniline film. Electrodeposition of the polyaniline film on the electrode was carried out galvanostatically in 1.0 M H₂SO₄ solution containing 0.10 M aniline.

FIG. 4A, trace 2, depicts a typical cyclic voltammogram of the detection electrode carrying the complex formed between the PNA capture molecule and the target nucleic acid molecule, after the incubation in the aniline/H₂O₂ solution. Obvious voltammetric activity was observed. Extensive washing and potential cycling thereafter produced no noticeable changes, revealing that the electroactive material (polyaniline) is robustly bound to the biosensor. While in a control experiment, non-complementary nucleic acid capture molecules failed to capture any GAPDH cDNA from the PCR mixture and thereby no horse-radish peroxidase labels were able to bind to the biosensor surface. Negligible redox activities in the potential window of −0.1 to 0.8 were observed, which are practically the same as those before the incubation in the aniline/H₂O₂ (FIG. 4 trace 3). These results clearly demonstrated that horse-radish peroxidase selectively interacted with the target nucleic acid molecule and the resulting nucleic acid/enzyme adduct catalysed the polymerisation of aniline and guides the deposition of polyaniline at the biosensor surface. When the complex formed between the PNA capture molecule and the target nucleic acid molecule is incubated in the mixture of aniline/H₂O₂ at pH 4.0, the protonated aniline molecules (pKₐ=4.9) are “concentrated” around the nucleic acid via electrostatic interaction between the protonated aniline molecules and phosphates in the nucleic acid. This high proton concentration around the nucleic acid provides a local environment of high acidity that permits polymerisation of aniline in a much less acidic medium than conventional electrochemical and chemical approaches, and facilitates a predominantly head-to-tail coupling and deters parasitic branching during polymerisation (Liu, et al., 1999, supra; Nagarajan, R., et al., 2001, supra). Thus, the deposition of polyaniline confirms and amplifies the sensing process of the analysed nucleic acid. To further elucidate the role of nucleic acid template in the biosensor, a neutral DNA analogue, a biotinlated PNA with sequence complementary to the PNA capture molecule was employed. No voltammetric activities were observed at both the biosensor and the control biosensor after hybridisation, incubation with avidin-horse-radish peroxidase conjugates, and with the aniline/H₂O₂ mixture, confirming that the hybridised nucleic acid indeed serves as templates in the deposition of polyaniline and there is little non-guided deposition of polyaniline.

As shown in FIG. 4A, in contrast to the electrodeposited polyaniline thin film, which had two pairs of redox peaks (FIG. 4A, trace 1), the horse-radish peroxidase-catalysed polyaniline displayed only one pair of redox peaks at ˜0.45 V. Similar results were obtained by Samuelson and co-workers for polyelectrolyte-templated polyaniline films (Liu, et al., 1999, supra; Nagarajan, R., et al., 2001, supra). These redox peaks have been assigned as the first redox process of polyaniline, leucoemeraldine/emeraldine. The absence of the second redox process is believed to be due to its high stability or the inability of the polyaniline to oxidize further to pernigraniline.

### Optimisation of Detection

The above voltammetric results confirmed that GAPDH is successfully detected in a PCR mixture with high specificity. Because of the inferior sensitivity of cyclic voltammetry relative to other voltammetric techniques, such as SWV, the more sensitive SWV was used in quantification experiments of this example, in order to maximize the performance of the method (FIG. 4B). However the sloppy background in 1.0 M H₂SO₄ was unfavourable in the development of an ultrasensitive detection method. It was found that the 0.10 M phosphate buffer of pH 4.0 generates a much lower background. Subsequent SWV experiments were therefore carried out in this buffer. For a successful adaptation of the present method of the invention in ultrasensitive nucleic acid assays, it is particularly advantageous, if the analytical signal is exclusively associated with the concentration of the target nucleic acid in a straightforward manner, such as a simple linear relationship.

The first possible cause of complexity lies in the heart of hybridisation, because hybridisation brings target nucleic acid to the biosensor, and as a direct consequence, increases the charge density on the biosensor surface. Any dependence of the deposition rate of polyaniline on the charge density would complicate the system, demanding complex algorithms to “decode” the information. Fortunately, our experiments showed that no obvious dependence is observed in the entire concentration range from femto- to nanomolar, agreeing well with previous study on the effect of polyelectrolyte on the polymerisation rate (Caramyshev et al., 2005, supra). This is probably due to the fact that horse-radish peroxidase only reacts with low molecular weight substrates. Constancy of the polyaniline deposition rate at various surface charge densities implies that the amount of the aniline involved in the polymerisation and deposition is simply proportional to the amount of horse-radish peroxidase at the biosensor surface and hence target nucleic acid concentration.

As mentioned earlier, a linear relationship between the SWV signal (amount of deposited polyaniline) and target nucleic acid concentration is advantageous in this system. In the case of polyaniline deposition, the deposition rate can be determined by one of the following: (i) mass-transport process of aniline in solution, (ii) horse-radish peroxidase-catalysed polymerisation process at the biosensor-solution interface, which is dependent on horse-radish peroxidase and H₂O₂, and (iii) nucleic acid-guided deposition. Under well-controlled conditions, when both the mass-transport and deposition process are much faster than the polymerisation process, the deposition rate is then solely controlled by the polymerisation process, which in turn, by the total amount of horse-radish peroxidase at the biosensor surface at a fixed H₂O₂ concentration, thereby by the concentration of target nucleic acid in solution. This sole horse-radish peroxidase-controlled process can be achieved by “speeding up” mass-transport since deposition rate is fast enough that it never becomes a rate-limiting process, evidenced by the independence of polyaniline deposition rate on the amount of polyelectrolyte (Caramyshev et al., 2005, supra). The mass-transport rate is directly proportional to the concentration of aniline. Higher mass-transport rates are obtainable when working with higher concentrations of aniline. As the aniline concentration increased, the signal rose rapidly and almost linearly at first, but then slowed until maximum sensitivity was reached (FIG. 5A). It was observed that the peak current is practically independent of aniline concentration at 20 mM, suggesting the enzymatic amplification does not appreciably deplete the concentration of monomeric aniline, the polyaniline deposition rate is now solely determined by the amount of horse-radish peroxidase brought to the biosensor surface. On the other hand, the sensitivity increased with increasing H₂O₂ concentration and a maximum was attained at 2.5 mM H₂O₂ (FIG. 5B). Higher H₂O₂ concentrations, e.g. 10 mM, deactivated horse-radish peroxidase almost instantly, reflected by a drastic decrease in peak current, consisting with the well-known mechanism of peroxidase catalysis.

FIG. 5C depicts the dependence of the SWV peak current on solution pH. The pH of the deposition solution was adjusted by adding a 0.10 M NaOH or a 0.10 M H₂SO₄ solution. It was found that the pH value of the polyaniline deposition medium has a profound effect on the SWV peak current of the biosensor. As illustrated in FIG. 5C, when the pH value was changed from 2.0 to 4.0, the oxidation current increased sharply, presumably due to the increase in horse-radish peroxidase stability and activity. The highest sensitivity was attained at pH 4.0. Further increase in pH resulted in a lower peak current, owing largely to the decreased aniline protonation and a lower proton concentration around the nucleic acid, although the optimal activity of horse-radish peroxidase is at pH ˜6.5. It is also possible that as the pH increases, the aniline alignment along the nucleic acid template is less than optimal for the head-to-tail polymerisation and the ortho branching may become more prevailing, resulting in the deposition of increasingly more electrochemically inactive polyaniline.

To leverage on the cumulative nature of the process for further improving the sensitivity of the biosensor, a series of SWV tests were conducted. A plot of the peak current at different incubation time is shown in FIG. 5D. In all cases, an immediate increase in the SWV peak current was observed due to the deposition of polyaniline. Interestingly, the sensitivity of the biosensor was proportional to the incubation time throughout, indicating that there is little activity loss of horse-radish peroxidase during incubation. However, there is a balance between high sensitivity and assay time. It was found that a 30 min-incubation period is sufficient to detect nucleic acid at femtomolar levels.

### Analytical Application in Nucleic Acid Assays

The applicability of the proposed biosensor in nucleic acid assays was tested on genomic samples. In the present example, three full-length genes, namely histone H4 (His4, 312 bp), breast cancer gene 1 (BRCA1, 5592 bp), and GAPDH (1008 bp) covering both high-/low-copy number and long/short genes, were used as calibration standards and were diluted to different concentrations with the hybridisation buffer before use. Analyte solutions with different concentrations of cDNA, ranging from 1.0 fM to 1.0 nM, were tested. For control measurements, nucleic acid capture molecules non-complementary to any of the genes were used in the biosensor preparation. As depicted in FIG. 6, the SWV data agreed well with the results obtained earlier and confirmed that all the three genes were successfully detected. The 30-min incubation period generated a dynamic range of 5.0 fM −2.0 pM with a relative standard derivation of <20% at 100 fM (20 duplicates) and a detection limit of ˜2.0 fM. It was noteworthy that the sensitivity is practically constant for both short and long genes, again confirming that the deposition process of polyaniline is solely controlled by the amount of horse-radish peroxidase on the biosensor surface. The specificity of the biosensor for the detection of target nucleic acid molecules was further evaluated by means of measurements, in which the PNA capture molecule (GAPDH gene) was complementary to the target nucleic acid molecule to different degrees. For this purpose, on detection electrodes a fully complementary nucleic acid capture molecule (see above, SEQ ID NO: 1), as well as nucleic acid capture molecules with one-, two-, and three-base-mismatches (highlighted in the sequences below) to GAPGH were immobilised. The GAPDH capture molecule with one mismatch was: 5′-TTACTCCTTAGAGGCCATGT-3′ (SEQ ID NO: 7); with two mismatches: 5′-TTATTCCTTGGAGGCCATAT-3′ (SEQ ID NO: 8); and with three mismatches: 5′-TTATTCCTTAGAGGCCATAT-3′ (SEQ ID NO: 9).

As shown in trace 1 in FIG. 7, the peak current increment for the biosensor coated with the fully complementary capture probes were in the range of 125-155 nA. If the fully complementary capture probes were replaced with mismatched probes (A⇄T or G⇄C mismatch in the middle of the capture probes, see sequences above), the current increment dropped by at least 70% when one base was mismatched, by ˜85% for two-base mismatched sequences, and it was indistinguishable from the background noise when three bases were mismatched, readily allowing selective detection of genes in a complex nucleic acid mixture and discrimination between the perfectly matched and mismatched sequences. Similar selectivity between perfectly complementary and mismatched sequences has been previously reported in other electrochemical nucleic acid assays (Zhang et al., 2003, supra; Xie, et al., 2004, supra; Gao, Z., Yang, Z., Anal. Chem. (2006) 78, 1470-1477; Lou, X., et al., Anal. Chem. (2005) 77, 4698-4705).

Detection of nucleic acid molecules according to the present invention may also be carried out in a method that omits a probe nucleic acid molecule, as explained above.

Specific detection of a target nucleic acid containing the GAPDH gene was also observed using both horse-radish peroxidase (FIG. 8) as a catalyst and after adding the initiator ammonium persulfate (FIG. 9). Again, only one redox peak was measured. Voltammograms of detection electrodes carrying the complex formed between the PNA capture molecule and the target nucleic acid molecule displayed significant activity for both catalysts (see trace 1 in FIG. 8 and FIG. 9), when compared to a control measurement using a non-complementary nucleic acid capture molecule (see trace 2 in FIG. 8 and FIG. 9). However, it should be noted that conditions as for the use of a probe nucleic acid were applied. Accordingly, no optimisation was carried out for this method in the present example, so that it can be expected that an enhanced signal to noise ratio is achievable, when compared to FIGS. 8 and 9.

### CONCLUSIONS

In summary, the above examples illustrate an electrochemical biosensor for nucleic acids based on a method of the present invention. The present examples utilizes the hybridised target nucleic acid as templates to guide the deposition of polyaniline catalysed by horse-radish peroxidase brought on the biosensor surface by the detection probes. The engagement of an integral factor greatly enhances the sensitivity of the biosensor. The dual-dependence (template and catalyst) of the signal amplification process substantially lowers non-hybridisation-related background noise since the non-specifically adsorbed nucleic acid or horse-radish peroxidase alone has little impact on the background signal.

1. A method of electrochemically detecting and/or quantifying a target nucleic acid molecule by means of a detection electrode, the method comprising:
(a) providing a detection electrode;
(b) immobilizing on said detection electrode a peptide nucleic acid (PNA) capture molecule, which has a nucleotide sequence that is at least partially complementary to at least a portion of the target nucleic acid molecule;
(c) contacting the electrode with a solution expected to comprise the target nucleic acid molecule;
(d) allowing the target nucleic acid molecule to hybridize to the PNA capture molecule on the electrode, thereby allowing the formation of a complex between said PNA capture molecule and said target nucleic acid molecule;
(e) adding a polymerisable positively chargeable precursor, wherein said polymerisable positively chargeable precursor has an electrostatic net charge that is complementary to the electrostatic net charge of the target nucleic acid molecule, such that (i) said polymerisable positively chargeable precursor associates to the complex formed between said PNA capture molecule and said target nucleic acid molecule, and (ii) the polymerisation of said precursor can be carried out by means of a suitable reactant molecule;
(f) adding a suitable reactant molecule, thereby initiating the polymerisation of said polymerisable positively chargeable precursor, wherein an electroconductive polymer is formed from the polymerisable precursor and this electroconductive polymer is associated with the complex formed between the PNA capture molecule and the target nucleic acid molecule;
(g) performing an electrochemical measurement at the detection electrode; and
(h) detecting and/or quantifying the presence of the target nucleic acid molecule based on the electrochemical measurement.

2. (canceled)

3. The method of claim 1, wherein the reactant molecule is an oxidant.

4. The method of claim 3, wherein the oxidant is at least one of a ruthenium tris(bipyridinium) complex, a persulfate, a peroxide, a chromate, a dichromate, a manganate, a permanganate, oxygen, ozone, a persulfate, a halogen, a chlorite, a chloride, a perchloride, a chlorate, a iodate, a nitrate, a sulfoxide and osmium tetroxide.

5. The method of claim 1, wherein (f) comprises exposing the reactant molecule to a suitable initiator.

6. The method of claim 5, wherein the initiator is one of a halogen molecule, an azo compound, a persulfate molecule, and a peroxide compound.

7. (canceled)

8. The method of claim 1, wherein (f) comprises exposing the reactant molecule to a suitable catalyst.

9. The method of claim 8, wherein the catalyst is selected from the group consisting of a metal chloride, a metal bromide, a metal sulphate and an enzyme.

10. The method of claim 9, wherein the reactant molecule is a substrate molecule for said catalyst.

11. The method of claim 9, wherein said catalyst is in solution or is coupled to a detection probe nucleic acid molecule, wherein said detection probe nucleic acid molecule is complementary to at least a portion of the target nucleic acid molecule.

12. The method of claim 11, wherein said detection probe nucleic acid molecule has a nucleic acid sequence of a length of about 5 to about 50 bp.

13. The method of claim 9, wherein said catalyst is an enzyme.

14. The method of claim 13, wherein said enzyme is a peroxidase enzyme or an oxidase enzyme.

15. The method of claim 1, wherein said PNA capture molecule has a nucleic acid sequence of a length of about 7 to about 30 bp.

16. (canceled)

17. A method of electrochemically detecting and/or quantifying a target nucleic acid molecule by means of a detection electrode, the method comprising:
(a) providing a detection electrode;
(b) immobilizing on said detection electrode a nucleic acid capture molecule, which has a nucleotide sequence that is at least partially complementary to at least a portion of the target nucleic acid molecule;
(c) contacting the electrode with a solution expected to comprise the target nucleic acid molecule;
(d) allowing the target nucleic acid molecule to hybridize to the nucleic acid capture molecule on the electrode, thereby allowing formation of a complex between said nucleic acid capture molecule and said target nucleic acid molecule;
(e) adding a polymerisable positively chargeable precursor, wherein said polymerisable positively chargeable precursor has an electrostatic net charge that is complementary to the electrostatic net charge of the target nucleic acid molecule, such that (i) said polymerisable positively chargeable precursor associates with the complex formed between said nucleic acid capture molecule and said target nucleic acid molecule, and (ii) the polymerisation of said precursor can be carried out by means of a suitable enzyme and a substrate molecule;
(f) adding a suitable substrate molecule;
(g) adding an enzyme attached to a detection probe nucleic acid molecule, wherein said probe nucleic acid molecule is at least partially complementary to at least a portion of the target nucleic acid molecule, wherein the detection probe hybridizes to a portion of the target nucleic acid different from the portion to which the capture nucleic acid molecule hybridizes, thereby (i) allowing the detection probe nucleic acid molecule to hybridise to the target nucleic acid molecule, and (ii) catalysing the polymerisation of said polymerisable positively chargeable precursor, wherein an electro conductive polymer is formed from the polymerisable precursor and this electroconductive polymer is associated with the complex formed between the nucleic acid capture molecule and the target nucleic acid molecule;
(h) performing an electrochemical measurement at the detection electrode, and;
(i) detecting and/or quantifying the presence of the target nucleic acid molecule.

18. (canceled)

19. The method of claim 17, wherein the substrate molecule is an oxidant.

20. The method of claim 19, wherein the oxidant is at least one of a persulfate, a peroxide, a chromate, a dichromate, a manganate, a permanganate, oxygen, ozone, a halogene, a chlorite, a chloride, a perchloride, a chlorate, a iodate, a nitrate, a sulfoxide and osmium tetroxide.

21. The method of claim 17, wherein said enzyme is a peroxidase enzyme or an oxidase enzyme.

22. The method of claim 17, wherein said detection probe nucleic acid molecule has a nucleic acid sequence of a length of about 5 to about 50 bp.

23. The method of claim 17, wherein said nucleic acid capture molecule has a nucleic acid sequence of a length of about 7 to about 30 bp.

24. (canceled)

25. The method of claim 17, wherein said nucleic acid capture molecule is one of DNA, RNA and PNA.

26. The method of claim 1, wherein said target nucleic acid molecule is DNA or RNA.

27. The method of claim 1, wherein said target nucleic acid molecule comprises a pre-defined sequence.

28. The method of claim 1, wherein the target nucleic acid molecule comprises at least one single-stranded region.

29. The method of claim 28, wherein said predefined sequence is a single-stranded region.

30. The method of claim 14, wherein said peroxidase enzyme is a haem peroxidase.

31. The method of claim 30, wherein said haem peroxidase is selected from the group consisting of horseradish peroxidase, cytochrome c peroxidase, glutathione peroxidase, myeloperoxidase, thyroid peroxidase, eosinophil peroxidase, lactoperoxidase, ascorbate peroxidase, peroxidasin, prostaglandin H synthase, E. coli catalase-peroxidase, M. tuberculosis catalase-peroxidase, Bacteroides fragilis catalase-peroxidase, lignin peroxidase, plant ascorbate peroxidase, Haem chloroperoxidase, manganese peroxidase, stigma specific peroxidase, Euphorbia characias latex peroxidase, Arthromyces ramosus peroxidase, sorghum grain peroxidase SPC4, soybean peroxidase, Phanerochaete chrysosporium manganese-dependent peroxidase, and lacrimal gland peroxidase.

32. The method of claim 14, wherein said oxidase enzyme is laccase.

33. (canceled)

34. The method of claim 1, wherein the positively chargeable precursor is an aromatic amine.

35. The method of claim 34, wherein said aromatic amine is selected from the group consisting of aniline, pyridineamine, pyrrole and imidazole.

36. The method of claim 34, wherein the target nucleic acid molecule is comprised in a solution of a pH selected in the range of about 1.7 to about 7.0, when said polymerisable positively chargeable precursor is added.

37. The method of claim 34, wherein the pH is brought to a value in the range of about 1.7 to about 7.0 after said polymerisable positively chargeable precursor has been added.

38. (canceled)

39. (canceled)

40. The method of claim 1, wherein the target nucleic acid molecule is comprised in a sample selected from the group consisting of a soil sample, an air sample, an environmental sample, a cell culture sample, a bone marrow sample, a rainfall sample, a fallout sample, a space sample, an extraterrestrial sample, a sewage sample, a ground water sample, an abrasion sample, an archaeological sample, a food sample, a blood sample, a serum sample, a plasma sample, a urine sample, a stool sample, a semen sample, a lymphatic fluid sample, a cerebrospinal fluid sample, a naspharyngeal wash sample, a sputum sample, a mouth swab sample, a throat swab sample, a nasal swab sample, a bronchoalveolar lavage sample, a bronchial secretion sample, a milk sample, an amniotic fluid sample, a biopsy sample, a nail sample, a hair sample, a skin sample, a cancer sample, a tumour sample, a tissue sample, a cell sample, a cell lysate sample, a virus culture sample, a forensic sample, an infection sample, a nosocomial infection sample, a production sample, a drug preparation sample, a biological molecule production sample, a protein preparation sample, a lipid preparation sample, a carbohydrate preparation sample, a solution of a nucleotide, a solution of polynucleotide, a solution of a nucleic acid, a solution of a peptide, a solution of a polypeptide, a solution of an amino acid, a solution of a protein, a solution of a synthetic polymer, a solution of a biochemical composition, a solution of an organic chemical composition, a solution of an inorganic chemical composition, a solution of a lipid, a solution of a carbohydrate, a solution of a combinatory chemistry product, a solution of a drug candidate molecule, a solution of a drug molecule, a solution of a drug metabolite, a suspension of a cell, a suspension of a virus, a suspension of a microorganism, a suspension of a metal, a suspension of metal alloy, a solution of a metal ion, and any combination thereof.

41. A kit for the electrochemical detection of a target nucleic acid molecule, said kit comprising
(a) a detection electrode,
(b) a PNA capture molecule, which has a nucleotide sequence that is at least partially complementary to at least a portion of the target nucleic acid molecule,
(c) a polymerisable positively chargeable precursor, wherein the electrostatic net charge of said polymerisable positively chargeable precursor is complementary to the electrostatic net charge of the target nucleic acid molecule, and
(d) a suitable reactant molecule.

42-53. (canceled)

54. A kit for the electrochemical detection of a target nucleic acid molecule, said kit comprising
(a) a detection electrode,
(b) a nucleic acid capture molecule, which has a nucleotide sequence that is at least partially complementary to at least a portion of the target nucleic acid molecule,
(c) a polymerisable positively chargeable precursor, wherein the electrostatic net charge of said polymerisable positively chargeable precursor is complementary to the electrostatic net charge of the target nucleic acid molecule,
(d) a substrate molecule, and
(e) an enzyme attached to a probe nucleic acid molecule, wherein said probe nucleic acid molecule is at least partially complementary to at least a portion of the target nucleic acid molecule.

55-66. (canceled)
